(19) United States
(12) Patent Application Publication
(10) Pub. No.: US 2026/0157676 A1
(43) Pub. Date: Jun. 11, 2026

(54) CUSTOMIZABLE, RECONFIGURABLE AND ANATOMICALLY COORDINATED LARGE-AREA, HIGH-DENSITY ELECTROMYOGRAPHY FROM DRAWN-ON-SKIN ELECTRODE ARRAYS

(71) Applicant: THE PENN STATE RESEARCH FOUNDATION, University Park, PA (US)

(72) Inventors: Cunjiang Yu, University Park, PA (US); Faheem Ershad, University Park, PA (US)

(21) Appl. No.: 19/127,353

(22) PCT Filed: Nov. 30, 2023
(86) PCT No.: PCT/US2023/081834

§ 371 Date: May 5, 2025

Related U.S. Application Data
(60) Provisional application No. 63/429,308, filed on Dec. 1, 2022.

(51) Int. Cl. A61B 5/296; A61B 5/259; A61B 5/265; A61B 5/268; A61F 2/58; A61F 2/68; A61F 2/70; A61F 2/72; B41M 1/12; B41M 1/38; B41M 3/00; B41M 5/00

(52) U.S. Cl. CPC A61B 5/296; A61B 5/259; A61B 5/265; A61B 5/268; A61F 2/583; A61F 2/586; A61F 2/72; A61B 2562/125; A61F 2002/6872; A61F 2002/705; B41M 1/12; B41M 1/38; B41M 3/006; B41M 5/0076

(57) ABSTRACT

Embodiments relate to a multielectrode array kit. The kit includes electrically conductive ink configured to adhere to a surface and form an electrode and an interconnect, an ink applicator configured to apply the electrically conductive ink to the surface, an insulative material applicator configured to apply an electrically insulative material to the surface, and an electrical contact configured to place the interconnect in electrical connection with a data acquisitioning system. Embodiments also relate to a method for performing electromyography. The method involves applying an electrically conductive ink to a surface of skin to form an electrode point, applying the electrically insulative material to the surface of skin, applying the electrically conductive ink to the insulative material to form an interconnect extending from an electrode point, and placing an electrical contact in electrical connection with the interconnect to facilitate electrical connection with a data acquisitioning system.

### CROSS-REFERENCE TO RELATED APPLICATIONS

This patent application is related to and claims the benefit of priority of U.S. provisional patent application No. 63/429,308, filed on Dec. 1, 2022, the entire contents of which is incorporated herein by reference.

### STATEMENT REGARDING FEDERALLY SPONSORED RESEARCH AND DEVELOPMENT

This invention was made with government support under Grant No. N00014-21-1-2480 awarded by the U.S. Navy/ONR, under Grant No. EB026175 awarded by the National Institutes of Health and under Grant No. CBET1936151 awarded by the National Science Foundation. The Government has certain rights in the invention.

### FIELD OF THE INVENTION

Embodiments relate to multielectrode arrays and methods of making and using the same.

### BACKGROUND OF THE INVENTION

Accurate anatomical matching for patient-specific electromyographic (EMG) mapping is crucial yet technically challenging in various medical disciplines. The fixed electrode construction of conventional multielectrode arrays (MEAs) makes it nearly impossible to match an individual's unique muscle anatomy. This mismatch between the MEAs and target muscles leads to missing relevant muscle activity, highly redundant data, complicated electrode placement optimization, and inaccuracies in classification algorithms.

### SUMMARY OF THE INVENTION

An exemplary embodiment can relate to a kit for preparation of a network of drawn-on sensors. The kit can include electrically conductive ink configured to adhere to a surface and form an electrode and an interconnect when applied to the surface. The kit can include an ink applicator configured to apply the electrically conductive ink to the surface. The kit can include an insulative material applicator configured to apply an electrically insulative material to the surface. The kit can include an electrical contact configured to place the interconnect in electrical connection with a data acquisitioning system. While exemplary embodiments describe the drawn-on ink as forming a sensor, it is understood that the drawn-on ink can be used to form a sensor, an electrode, an electronic device, a component of an electronic device, etc.

In some embodiments, the ink applicator can be a pen, a brush, a dispensing device, and/or a printer device. The insulative material applicator can be a pen, a brush, a dispensing device, and/or a printer device.

In some embodiments, the surface can be skin of an animal, skin of a human, or a surface of artificial or synthetic skin.

In some embodiments, the kit can include a stencil configured to be placed against the surface and guide application of the electrically conductive ink and/or the insulative material.

In some embodiments, the kit can include conductive glue configured to adhere the electrical contact to the surface.

In some embodiments, the electrical contact includes an electrically conductive wire, an electrically conductive film, and/or an electrically conductive pad.

In some embodiments, the electrical contact can include an anisotropic material.

In some embodiments, the kit can include the data acquisitioning system.

In some embodiments, the electrically conductive ink can include an Ag/poly(3,4-ethylenedioxythiophene)-poly(styrenesulfonate) (“Ag-PEDOT:PSS”) composite. The electrically insulative material can include a water-based acrylic emulsion.

An exemplary embodiment can relate to a method for fabricating an electrode ink. The method can involve preparing a poly(3,4-ethylenedioxythiophene)-poly(styrenesulfonate) (“PEDOT:PSS”) solution. The method can involve adding Ag flakes to the solution in a 1:2 weight ratio of Ag flakes to PEDOT:PSS solution to form a Ag-PEDOT:PSS composite. The method can involve stirring and/or agitating the Ag-PEDOT:PSS composite.

An exemplary embodiment can relate to a method for generating a sensor network. The method can involve applying an electrically conductive ink to a surface of skin to form an electrode point. The method can involve applying the electrically insulative material to the surface of skin. The method can involve applying the electrically conductive ink to the insulative material to form an interconnect extending from an electrode point. The method can involve placing an electrical contact in electrical connection with the interconnect, wherein the electrical contact is configured to be placed in electrical connection with a data acquisitioning system.

In some embodiments, the method can involve placing the electrical contact in electrical connection with the data acquisitioning system.

In some embodiments, the method can involve monitoring, measuring, and/or sensing electrical activity of the electrode. The electrical activity can include voltage, a change in voltage, current, a change in current, impedance, and/or a change in impedance.

In some embodiments, the method can involve performing electromyography; and/or determining a movement, a gesture, a muscle activation, and/or a muscle relaxation based on the electrical activity.

In some embodiments, the method can involve measuring muscle response or electrical activity in response to a nerve's stimulation of a muscle.

In some embodiments, the method can involve detecting a neuromuscular abnormality.

In some embodiments, the method can involve translating the movement, the gesture, the muscle activation, and/or the muscle relaxation to an actuation signal configured to be transmitted to a prosthetic, a robotic prosthetic, or a gesture-controlled robot.

In some embodiments, the method can involve allowing or forcing flexure of the skin.

In some embodiments, the flexure of the skin can induce strain on the electrically conductive ink to cause elastic deformation of the electrically conductive ink.

In some embodiments, the method can involve forming a multielectrode array or a network of sensors on the surface of skin by applying plural electrodes and plural interconnects in an arrangement.

In some embodiments, the skin is animal skin, human skin, or artificial or synthetic skin.

An exemplary embodiment can relate to the method for creating at least one sensor on skin. The method can involve applying an electrically conductive ink to a surface of skin to form an electrical circuit. The skin can be animal skin, human skin, or artificial or synthetic skin.

In some embodiments, signals generated from the electrical circuit can be artifact-free.

Further features, aspects, objects, advantages, and possible applications of the present invention will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present innovation will be more apparent from the following more particular description thereof, presented in conjunction with the following drawings. Like reference numbers used in the drawings may identify like components.

FIGS. 1A, 1B, 1C, 1D, and 1E show exemplary embodiments of Drawn-On-Skin (DoS) electronics configured as high-density, muscle-specific multielectrode arrays (MEAs). FIG. 1 A is an exemplary kit for preparation of a multielectrode array. FIG. 1B illustrates DoS MEA drawing process showing customized positions of electrodes and interconnects being fabricated directly on the skin with a ballpen and DoS Ag-PEDOT:PSS ink (scale bar=5 mm). FIG. 1C shows deformability of DoS MEAs on skin when at 0% strain, 10% stretching, and 10% compressing (scale bar=1 cm). FIG. 1D shows large-area, high-density DoS MEA and interconnection pattern on the trapezius muscle of a human subject (scale bar=1 cm). FIG. 1E shows DoS MEAs customized to biceps brachii and forearm muscles (scale bar=5 cm), triceps brachii (scale bar=5 cm), and facial muscles, including the zygomaticus and risorius muscles (scale bar=2 cm).

FIGS. 2A, 2B, 2C, 2D, 2E, and 2F show DoS MEA skin-electrode impedance characterization. FIG. 2A shows a photograph of the DoS MEA fabricated on the forearm of a subject in a 3×5 (row×column) array, 3 mm electrode diameter, and 5 mm interelectrode spacing (scale bar=2 cm). FIG. 2B shows average normalized skin-electrode impedance over time from all subjects at EMG relevant frequencies after drawing all electrodes of the DoS MEAs. Data are presented as mean±s.d. FIG. 2C shows average normalized skin-electrode impedance spectrum from all subjects after adding additional electrodes to the DoS MEAs at 0, 20, and 40 min. Data are presented as mean±s.d. FIG. 2D shows images of the DoS MEA (top), stretchable Au MEA, and PEDOT:PSS on the forearms of subjects. The labeling of ‘A-D’ and ‘1-3’ in the DoS MEA camera image indicates the rows and columns which correspond to the heatmaps (scale bar=5 mm). FIG. 2E shows average skin-electrode impedance heatmaps from each of the three MEAs at different measurement frequencies (50, 250, 500 Hz). FIG. 2F shows EMG data recorded with three flexions of the flexor group of muscles in the forearm using the DoS MEA. The initial flexion was done without any skin deformation to the MEA. The following two flexions were performed with skin deformation, first stretching the skin around the edge of the DoS MEA and then compressing the skin.

FIGS. 3A, 3B, 3C, 3D, and 3E show high-density DoS MEA usage for muscle activity assessments. FIG. 3A shows high-density DoS MEA on the flexor muscle group of a subject, inset shows the orientation of the layout (‘A-D’ for rows and ‘1-8’ for columns, scale bar=1 cm) for motor unit propagation mapping and innervation zone localization (scale bar=2 cm). FIG. 3B shows a propagation map of a single row of the high-density DoS MEA. The change in the inflection of the wave in the third trace from the bottom (indicated by the red star) denotes the innervation zone, and the red arrows indicate the characteristic ‘V’ pattern indicating propagation of the motor unit action potential in different directions from the innervation zone. FIG. 3C shows complete propagation maps for the entire DoS MEA and innervation zone band indicated by the red stars connected with dotted lines. FIG. 3D shows a setup for DoS MEA and conventional FPC grid comparison of EMG measurement during seated resistance band bicep curls. The labeling of the rows is used to calculate average EMG signals across each of the rows of the respective MEAs for signal quality examination. FIG. 3E shows signal-to-noise ratios of averaged EMG signals from each row of the DoS MEA and FPC grid during 30 min of exercise.

FIGS. 4A, 4B, and 4C show reconfigurable DoS MEAs implemented with a conventional grid for muscle activity localization during hand flexions. FIG. 4A shows Vᵣₘₛ heatmaps of EMG signals acquired from DoS electrodes arranged in 8×2 arrays beside the FPC grid to cover the forearm in lateral and medial directions to confine the center of activity during four different hand gestures including (1) hand close; (2) thumb, index, middle flexion; (3) middle, ring flexion; and (4) ring, little flexion. FIG. 4B shows Vᵣₘₛ heatmaps of EMG signals acquired from DoS electrodes arranged in 2×8 arrays beside the FPC grid to cover the forearm in proximal and distal directions. FIG. 4C shows Vᵣₘₛ heatmaps of EMG signals acquired from DoS electrodes arranged in a 4×8 array beside the FPC grid to cover the forearm in the distal direction.

FIGS. 5A, 5B, 5C, 5D, 5E, and 5F show subject-customized DoS MEAs for finger gesture classification and prosthetic hand control. FIG. 5A shows custom stencils of linear eight-electrode arrays placed as four rows (to form a 4×8 array) around the varying circumferences (indicated by the 4 labeled positions) of the forearm of a subject (scale bar=2 cm). FIG. 5B shows a camera image of a completed and customized DoS MEA over the flexor and extensor groups for flexion and extension-based finger gesture classification (scale bar=2 cm). FIG. 5C shows Vᵣₘₛ feature maps of different gestures on lateral views of the forearm. FIG. 5D shows a confusion matrix from a linear discriminant analysis classifier after offline analysis of EMG data obtained with two FPC grids having 128 channels. These grids only covered a portion of the circumference of the forearm. The numbers on the axes correspond to the labels in the feature maps above. FIG. 5E shows a confusion matrix from a linear discriminant analysis classifier after offline analysis of EMG data obtained with DoS MEAs having 32 channels. The DoS MEAs covered the entire circumference of the forearm. The numbers on the axes correspond to the labels in the feature maps above. FIG. 5F shows near real-time control of a prosthetic hand by a human subject wearing the customized DoS MEA to mimic ring, little flexion (left, scale bar=5 cm); thumb, index, middle flexion (middle, scale bar=5 cm); and hand closed (right, scale bar=5 cm).

FIGS. 6A, 6B, 6C, and 6D show DoS MEA fabrication. FIG. 6A shows a tape based stencil laminated onto the forearm of the human subject. FIG. 6B shows DoS conductive ink drawn into the positions for the electrodes in the MEA. FIG. 6C shows brushing of the acrylic-based insulating material (Pros-Aide, ADM Tronics) onto the interconnect regions of the MEA. FIG. 6D shows after a few minutes of drying, the DoS conductive ink was drawn on top of the insulating material and left to dry for a few more minutes. Scale bars=1 cm.

FIGS. 7A and 7B show data acquisition approaches from customized DoS electrodes and MEAs. FIG. 7A shows electrode collar adhesive used to secure the stainless steel wires from the data acquisition system directly to the DoS electrode (scale bar=1 cm). Below is a schematic of the cross section. FIG. 7B shows conductive wire glue (Wire Glue, American Science and Surplus) used to fix the stainless-steel wire on the interconnects of DoS MEAs (scale bar=2 cm). Zoom in shown on the image on the right (scale bar=2 mm). The glue was placed in two positions to clamp the wire down and then DoS conductive ink was used to cover the entire exposed portion of the wire and dried wire glue.

FIG. 8 shows a data acquisition approach from DoS MEAs with prefabricated interconnections. The interconnection films were laminated onto the interconnects of the DoS MEAs. Since the DoS MEA did not have a symmetrical arrangement in this instance, the interconnection film was fabricated so that it could be laminated to either side of the DoS MEA. Scale bar=1 cm.

FIG. 9 shows a fabrication process for the stretchable Au MEA.

FIG. 10 shows a fabrication process for the printed PEDOT:PSS MEAs. The image on the left (scale bar=1 cm) shows the PEDOT:PSS droplet forming at the tip of the needle attached to a syringe of a custom-built pneumatic extrusion printer. The image on the right (scale bar=1 cm) shows the printing process.

FIG. 11 shows detailed geometrical dimensions of the MEAs, wherein the top shows dimensions of the stretchable Au MEA (inset shows dimensions of the serpentine pattern for the interconnects) and the bottom shows dimensions of the DoS and PEDOT:PSS MEAs.

FIG. 12 shows a comparison of the normalized skin-electrode impedance between the DoS, Au, PEDOT:PSS MEAs, and FPC grid. Data are presented as mean±s.d.

FIG. 13 shows custom connection scheme to capture data from the FPC grid. The customized adapter was custom-made so that the grid could be used with an Intan Recording Controller and amplifier. Scale bar=5 cm.

FIG. 14 shows formalized skin-electrode impedance from a subset of electrodes in the FPC grid. Scale bar=5 mm.

FIG. 15 (images A, B, C, d, E, F, and G) shows the effect of skin deformation-induced motion on DoS and wearable MEAs. Image A is a zoomed-in view of EMG data around the duration of skin deformation recorded with the DoS MEA. Image B is EMG data recorded with three flexions of the flexor group of muscles in the forearm using the stretchable Au MEA. The initial flexion was done without any skin deformation to the MEA. The following two flexions were performed with skin deformation, first stretching the skin around the edge of the stretchable Au MEA and then compressing the skin. Image C is a zoomed-in view of EMG data around the duration of skin deformation recorded with the stretchable Au MEA. Image D is EMG data recorded with three flexions of the flexor group of muscles in the forearm using the printed PEDOT:PSS MEA. The initial flexion was done without any skin deformation to the MEA. The following two flexions were performed with skin deformation, first stretching the skin around the edge of the DoS MEA and then compressing the skin. Image is a zoomed-in view of EMG data around the duration of skin deformation recorded with the printed PEDOT:PSS MEA. Image F is EMG data recorded with three flexions of the flexor group of muscles in the forearm using the FPC Grid. The initial flexion was done without any skin deformation to the MEA. The following two flexions were performed with skin deformation, first stretching the skin around the edge of the FPC Grid and then compressing the skin. Image G is a zoomed-in view of EMG data around the duration of skin deformation recorded with the FPC Grid.

FIG. 16 (graphs A, B, C, and D) shows Fast Fourier Transform data from each of the arrays. Each graph shows the FFT data from the EMG signal during the initial contraction of the skin-deformation induced motion artifacts comparison from a single subject. Graph A is the average data from all channels recorded with the DoS MEA; graph B is the average data from all channels recorded with the stretchable Au MEA; graph C is the average data from all channels recorded with the PEDOT:PSS MEA; and graph D is the average data from all channels recorded with the FPC grid.

FIG. 17 shows detailed geometrical dimensions of the FPC grid.

FIGS. 18A, 18B, 18C, 18D, and 18E show a layout of a Dos MEA and propagation maps for each row from the high-density DoS MEA. FIG. 18A shows a layout of the DoS MEA with the rows labeled with letters and the columns labeled with numbers. Note that the propagation maps were constructed using a bipolar method, where the difference of the neighboring electrodes in one row was used to find motor units instead of using the individual data channel data. Scale bar=1 cm. FIG. 18B shows a propagation map of row ‘A’ of the high-density DoS MEA. The change in the inflection of the wave in the third trace from the bottom (indicated by the red star) denotes the innervation zone and the red arrows indicate the characteristic ‘V’ pattern indicating propagation of the motor unit action potential in different directions from the innervation zone. FIG. 18C shows a propagation map of row ‘B’ of the high-density DoS MEA.

FIG. 18D shows a propagation map of row ‘C’ of the high-density DoS MEA. FIG. 18 E shows a propagation map of row ‘D’ of the high-density DoS MEA.

FIG. 19 shows placement of FPC grid above the forearm flexors for the motor unit detection comparison. Scale bar=1 cm.

FIG. 20 show a setup for evaluating the quality of the EMG signals during substantial muscle movement underneath the skin. The image on the left shows the DoS MEA on the right arm of the subject and the FPC grid on the left arm. Over several repetitions of the exercise, the grid started to delaminate (indicated by the red arrow, scale bar=10 cm). The image on the right (scale bar=1 cm) shows the connection of the DoS MEA to the data acquisition system using the tape to connect stainless-steel wires directly to the DoS electrodes.

FIG. 21 shows various finger gestures performed throughout this work for multiple experiments.

FIG. 22 shows a reconfigured DoS MEA used in conjunction with the FPC grid. The image above is of arrangement 3, with all the DoS electrodes positioned on one side of the FPC grid. Scale bar=1 cm.

FIG. 23 shows custom stencils for each subject for the finger gesture classification experiment. Each subject has a unique circumference of their forearm and the stencils are linear arrays placed circumferentially on 4 positions of the forearm, each position spaced ˜2 cm apart.

FIG. 24 shows medial view of forearm excitation maps for each finger gesture. The top row shows the Vᵣₘₛ, maps from the flexion gestures and the bottom row shows the Vᵣₘₛ maps from the extension gestures.

FIG. 25 shows FPC grids placement around the forearm for the finger gesture classification experiment. Scale bar=2 cm.

FIGS. 26A, 26B, 26C, and 26D show principal component analysis of EMG features. FIG. 26A shows principal component map with the first three components obtained from data recorded with the customized DoS MEA. The colors in the color bar, from bottom to top represent gestures 1-8 which correspond to (1) Hand closed; (2) Thumb, index, middle flexion; (3) Middle, ring flexion; (4) Ring, little flexion; (5) Hand opened; (6) Thumb, index, middle extension; (7) Middle, ring extension; and (8) Ring, little extension. FIG. 26B show percentage of variance based on each principal component identified from data recorded with the customized DoS MEA. FIG. 26 C show principal component map with the first three components obtained from data recorded with the two FPC grids. FIG. 261) shows percentage of variance based on each principal component identified from data recorded with the two FPC grids.

### DETAILED DESCRIPTION OF THE INVENTION

The following description is of exemplary embodiments that are presently contemplated for carrying out the present invention. This description is not to be taken in a limiting sense, but is made merely for the purpose of describing the general principles and features of the present invention. The scope of the present invention is not limited by this description.

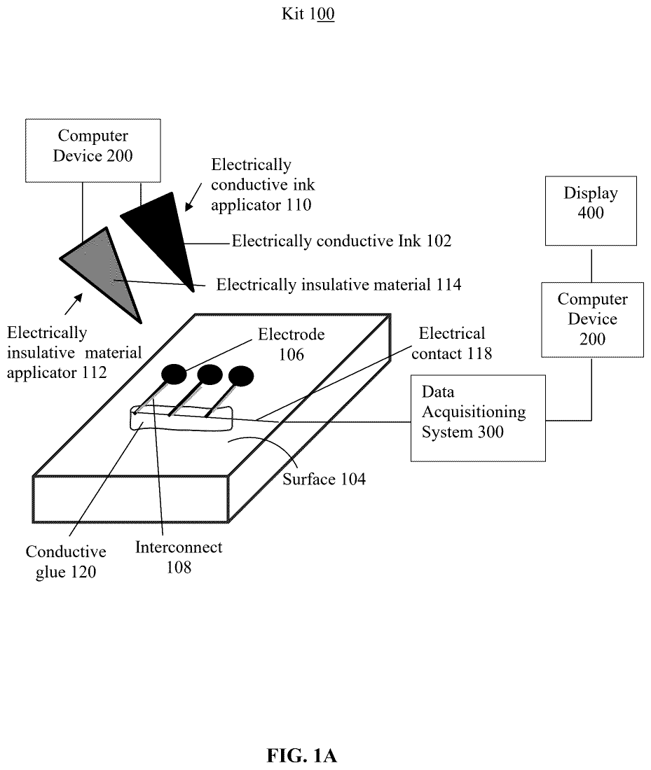

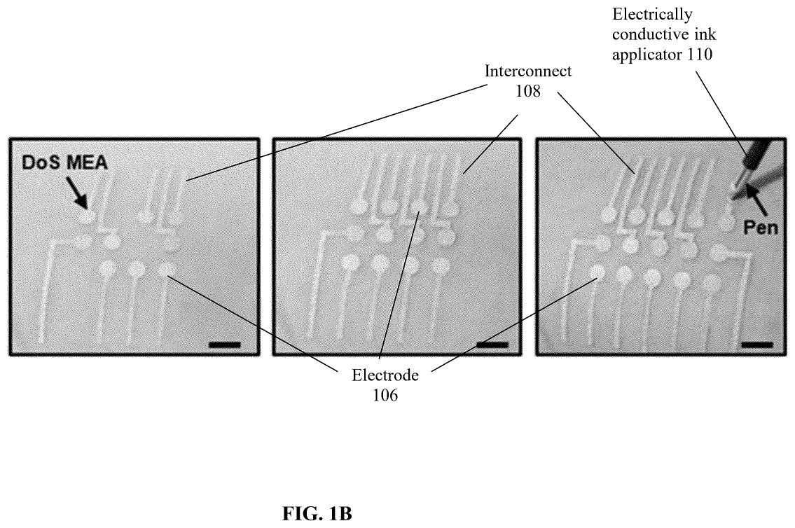

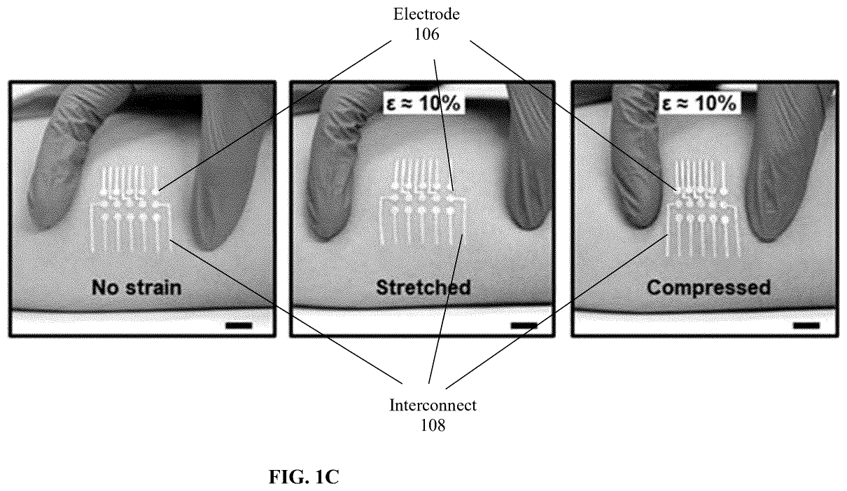

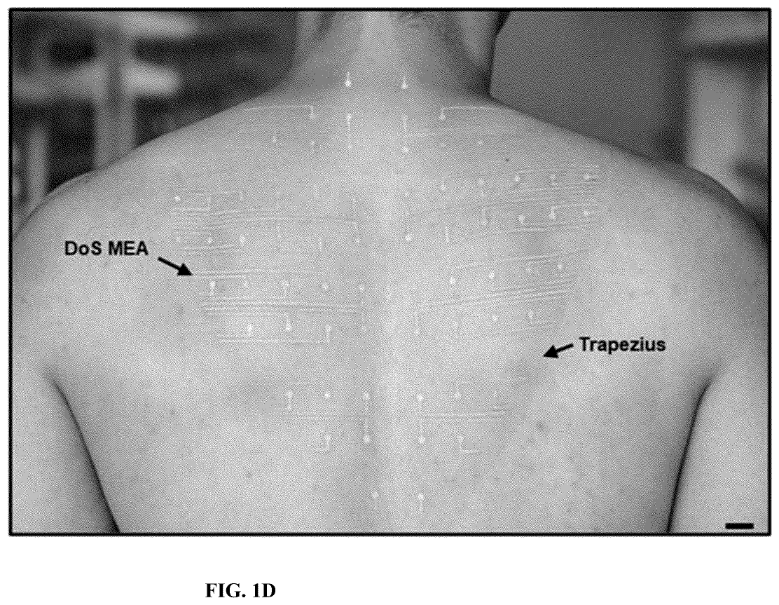

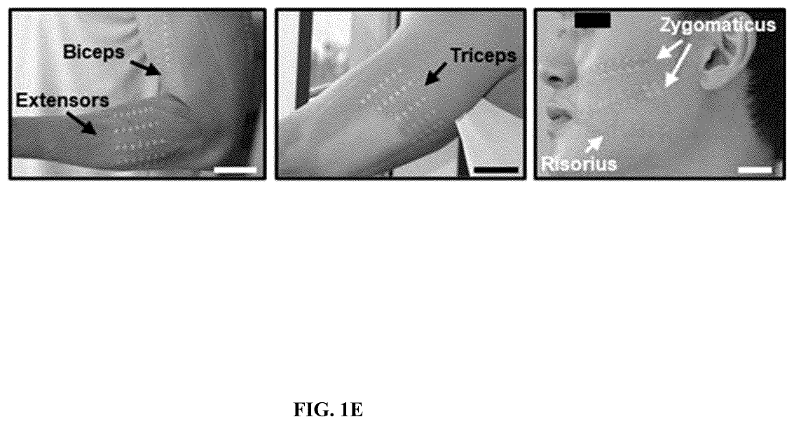

Referring to FIG. 1A, an exemplary embodiment can relate to a kit 100 for preparation of a network of drawn-on sensors (which can include a multielectrode array, for example). The kit can include electrically conductive ink 102. The electrically conductive ink 102 can be configured to adhere to a surface 104, and form an electrode 108 and an interconnect 108 when applied to the surface 104. It is contemplated for the surface 104 to be skin of an animal, skin of a human, artificial skin, synthetic skin, etc. It is also contemplated for the electrically conductive ink 102 to be applied to form a pattern of one or more electrodes 108 and one or more interconnects 108 on the surface 104. As will be explained herein, the multielectrode array can be used for monitoring, measuring, sensing, etc. neuromuscular activity. For instance, the when the ink 102 is applied to skin, neuromuscular activity of the animal can generate electrical activity in the electrode 108 that is representative of the neuromuscular activity.

The kit 100 can include an ink applicator 110. The ink applicator 110 can be configured to apply the electrically conductive ink 102 to the surface 104. The ink applicator 110 can be a pen, a brush, a dispensing device (e.g., spray device, plunger-style dispenser, etc.) a printer device (e.g., a 3D printer, inkjet printer, drop-on-demand printer, etc.), etc. The electrically conductive ink can be an Ag/poly(3,4-ethylenedioxythiophene)-poly(styrenesulfonate) (“Ag-PEDOT:PSS”) composite, for example. Other inks can include liquid metals, graphite-based inks, silver (Ag) only based inks, hydrogels, etc.

The kit 100 can include an electrically insulative material 114. The kit 100 can also include an insulative material applicator 112. The insulative material applicator 112 can be configured to apply the electrically insulative material 114 to the surface 104. The insulative material applicator 112 can be a pen, a brush, a dispensing device (e.g., spray device, plunger-style dispenser, etc.) a printer device (e.g., a 3D printer, inkjet printer, drop-on-demand printer, etc.), etc. The electrically insulative material 114 can include a water-based acrylic emulsion. Other electrically insulative material can include liquid bandages, liquid adhesives, etc.

As will be explained herein, the electrically conductive ink 102 is applied directly to the surface 104 when forming the electrode(s) 108, whereas for the formation of the interconnects 108 the electrically insulative material 114 is first applied to the surface 104 and the electrically conductive ink 102 is applied on top of the electrically insulative material 114.

Depending on the type of ink applicator 110 and insulative material applicator 112 used, these devices can also have processors and associated memory to allow the applicator(s) to operate automatically or semi-automatically. For instance, these applicators may be 3D printing type applicators. The processors of these applicators can include software, hardware, firmware, etc. that facilitate automatic or semi-automatic application the electrically conductive ink 102 or electrically insulative material 114 to the surface 104 via a programmed algorithm(s) so as to generate a pattern on the surface. The pattern can be one or more arrays, motifs, designs, arrangements, etc. of electrodes 108 and interconnects 108 that are optimal in monitoring, measuring, sensing, etc. neuromuscular activity. Optimization can include factors such as producing most accurate neuromuscular activity, using the least computational resources, providing the quickest processing time, etc. Optimization can also include use of objective functions, cost functions, etc. to determine the best trade-offs between factors so as to meet a particular design objective. The pattern of the electrodes and interconnects, placement and orientation of the electrodes and interconnects on the skin, geometric shapes and sizes of the electrodes and interconnects, the design objectives, the optimization factors, etc. can be determined by program logic, algorithms, artificial intelligence, machine learning, etc. In addition, or in the alternative, these can be determined by user-input via a computer device 200 that is in communication with applicator(s).

In some embodiments, the kit 100 can include one or more stencils 116. The stencil 116 can be configured to be placed against the surface 104 and guide application of the electrically conductive ink and/or the insulative material. Thus, the stencil 116 can have the optimized pattern. The optimized pattern can be determined by a computer device 200 using the techniques discussed herein, wherein a machine (stamp machine, laser cutting machine, etc.) can create the stencil 116. There can be more than one stencil 116, wherein one stencil 116 may be optimized for a certain portion of the skin whereas another stencil 116 is optimized for another portion of the skin. As another example, one stencil 116 may be optimized for one design criterium whereas another stencil 116 may be optimized for another design criterium.

The kit can include an electrical contact 118. The electrical contact 118 can be configured to place the interconnect 108 in electrical connection with a data acquisitioning system 300. The electrical contact 118 can be an electrically conductive wire, an electrically conductive film, an electrically conductive pad, etc. The electrical contact 118 can be an anisotropic material so as to allow electrical current to flow in one direction but not in other directions (e.g., allow flow of electrical current from the interconnect 108 to the data acquisitioning system 300 but prevent electric current from flowing from one electrode 108 to another electrode 108). The kit 100 can also include conductive glue 120 configured to adhere the electrical contact 118 to the surface 104. The conductive glue 120 can be applied via a brush applicator, a spray applicator, etc. The conductive glue 120 can be a composite of graphite, polyvinyl acetate, and water, for example.

In some embodiments, the kit 100 can include the data acquisitioning system 300. The data acquisitioning system 300 is configured to monitor, measure, sense, etc. electrical activity of the electrode 108. The electrical activity can include voltage, a change in voltage, current, a change in current, impedance, a change in impedance, etc. For instance, when the ink 102 is applied to skin, neuromuscular activity of the animal can generate electrical activity in the electrode 108. This electrical activity is transmitted to the data acquisitioning system 300 via the interconnect(s) 108 and electrical contact(s) 118. The data acquisitioning system 300 converts the electrical activity into signals. These signals can be stored and/or further processed into data structures that are representative of the neuromuscular activity. The data acquisitioning system 300 can include sensors, processors, memory, hardware, firmware, software, etc. to facilitate data acquisition, processing, etc.

It is understood that the network of drawn-on sensors can include other sensors, electronics, electrical components, such as physiological sensors, metabolic sensors, etc. for example. These can be placed within the circuit formed by the ink. Thus, the network of drawn-on sensors can be in physical or electrical contact with an EKG sensor, accelerometer sensor, a motion sensor, etc. Any of these sensors can be placed in the circuit or be separate from the circuit but placed in electrical connection or communication with a component of the circuit. In this regard, any of these sensors may include processors, transmitters, etc. to facilitate data transmission. The data from these sensors can be used to confirm, augment, etc. the drawn-on sensor data. For instance, embodiments can use sensor fusion or other techniques to improve performance, create efficiencies, provide redundancies, etc. As another example, the drawn-on sensor circuit can be placed in electrical connection or communication with hardware (e.g., a computer device 200, a data acquisitioning system 300, etc.) to measure EKG, motion, acceleration, etc. The hardware can acquisition data from the conductive ink circuit that is representative of EKG, motion, acceleration, etc.

Any of the processors disclosed herein can be part of or in communication with a machine (e.g., a computer device, a logic device, a circuit, an operating module (hardware, software, and/or firmware), etc.). The processor can be hardware (e.g., processor, integrated circuit, central processing unit, microprocessor, core processor, computer device, etc.), firmware, software, etc. configured to perform operations by execution of instructions embodied in computer program code, algorithms, program logic, control, logic, data processing program logic, artificial intelligence programming, machine learning programming, artificial neural network programming, automated reasoning programming, etc.

Any of the processors disclosed herein can be a scalable processor, a parallelizable processor, a multi-thread processing processor, etc. The processor can be a computer in which the processing power is selected as a function of anticipated network traffic (e.g., data flow). The processor can include any integrated circuit or other electronic device (or collection of devices) capable of performing an operation on at least one instruction, which can include a Reduced Instruction Set Core (RISC) processor, a Complex Instruction Set Computer (CISC) microprocessor, a Microcontroller Unit (MCU), a CISC-based Central Processing Unit (CPU), a Digital Signal Processor (DSP), a Graphics Processing Unit (GPU), a Field Programmable Gate Array (FPGA), etc. The hardware of such devices may be integrated onto a single substrate (e.g., silicon “die”), or distributed among two or more substrates. Various functional aspects of the processor may be implemented solely as software or firmware associated with the processor.

The processor can include one or more processing or operating modules. A processing or operating module can be a software or firmware operating module configured to implement any of the functions disclosed herein. The processing or operating module can be embodied as software and stored in memory, the memory being operatively associated with the processor. A processing module can be embodied as a web application, a desktop application, a console application, etc.

The processor can include or be associated with a computer or machine readable medium. The computer or machine readable medium can include memory. Any of the memory discussed herein can be computer readable memory configured to store data. The memory can include a volatile or non-volatile, transitory or non-transitory memory, and be embodied as an in-memory, an active memory, a cloud memory, etc. Examples of memory can include flash memory, Random Access Memory (RAM), Read Only Memory (ROM), Programmable Read only Memory (PROM), Erasable Programmable Read only Memory (EPROM), Electronically Erasable Programmable Read only Memory (EEPROM), FLASH-EPROM, Compact Disc (CD)-ROM, Digital Optical Disc DVD), optical storage, optical medium, a carrier wave, magnetic cassettes, magnetic tape, magnetic disk storage or other magnetic storage devices, or any other medium which can be used to store the desired information and which can accessed by the processor.

The memory can be a non-transitory computer-readable medium. The term “computer-readable medium” (or “machine-readable medium”) as used herein is an extensible term that refers to any medium or any memory, that participates in providing instructions to the processor for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, transmission media, etc. The computer or machine readable medium can be configured to store one or more instructions thereon. The instructions can be in the form of algorithms, program logic, etc. that cause the processor to execute any of the functions disclosed herein.

Embodiments of the memory can include a processor module and other circuitry to allow for the transfer of data to and from the memory, which can include to and from other components of a communication system. This transfer can be via hardwire or wireless transmission. The communication system can include transceivers, which can be used in combination with switches, receivers, transmitters, routers, gateways, wave-guides, etc. to facilitate communications via a communication approach or protocol for controlled and coordinated signal transmission and processing to any other component or combination of components of the communication system. The transmission can be via a communication link. The communication link can be electronic-based, optical-based, opto-electronic-based, quantum-based, etc. Communications can be via Bluetooth, near field communications, cellular communications, telemetry communications, Internet communications, etc.

Transmission of data and signals can be via transmission media. Transmission media can include coaxial cables, copper wire, fiber optics, etc. Transmission media can also take the form of acoustic or light waves, such as those generated during radio-wave and infrared data communications, or other form of propagated signals (e.g., carrier waves, digital signals, etc.).

Any of the processors can be in communication with other processors of other devices (e.g., a computer device, a computer system, a laptop computer, a desktop computer, etc.). For instance, the processor of the data acquisitioning system 300 can be in communication with the processor of a computer device 200, wherein the processor of the computer device 200 can be in communication with a processor of a display 400. The data acquisitioning system 300 can transmit the electrical activity signals to the computer device 200 for further processing so that the computer device 200 caused the display 400 to display data representations of the signals (e.g., textual, graphical, graphical user interface, etc. display of the data). Any of the processors can have transceivers or other communication devices/circuitry to facilitate transmission and reception of wireless signals. Any of the processors can include an Application Programming Interface (API) as a software intermediary that allows two or more applications to talk to each other. Use of an API can allow software of the processor of the system 300 to communicate with software of the processor of the other device(s).

An exemplary embodiment can relate to a method for fabricating the electrode ink 102. The method can involve preparing a poly(3,4-ethylenedioxythiophene)-poly(styrenesulfonate) (“PEDOT:PSS”) solution. Ag flakes can then be added to the solution in a 1:2 weight ratio of Ag flakes to PEDOT:PSS solution to form an Ag-PEDOT:PSS composite. Ag-PEDOT:PSS composite can then be stirred or agitated for a predetermined amount of time.

An exemplary embodiment can relate to a method for generating a sensor network. The method can involve applying an electrically conductive ink 102 to a surface of skin to form one or more electrode points (e.g., one or more electrodes 108). For the formation of electrode(s) 108, the electrically conductive ink 102 is applied directly to the skin. This continues until a desired pattern or array of electrodes 108 are formed. For the formation of the interconnects 108, electrically insulative material 114 is first applied to the skin—i.e., the interconnects 108 will be formed by applying electrically conductive ink 102 on top of the electrically insulative material 114. Each interconnect 108 can form a connection between two or more electrodes 108—e.g., each interconnect 108 is electrically conductive ink 102 extending from an electrode 108 so as to run along and on top of a strip/path/area of electrically insulative material 114 and terminates at another electrode 108. While it is contemplated for the interconnect 108 to extend between at least two electrode 108, there may be some patterns in which the interconnect 108 merely extends from an electrode 108 without connecting to another electrode 108—i.e., the interconnect 108 can extend from an electrode 108 and terminate without having a connection or extend from an electrode 108 an connect to an electrical contact 118. The formation of the array of electrodes 108 and interconnects 108 can be such that all electrodes 108 are formed before the interconnect 108 layout (the pattern of electrically conductive material on top of the electrically insulative material) is formed, the interconnect layout is formed before the electrodes 108, each interconnect 108 is formed as each electrode 108 is formed, etc. Depending on the application, the formation of the array of electrode 108 and interconnects 108 can include the use of one or more stencils 116.

After the pattern of electrodes 108 and interconnects 108 are formed, one or more electrical contact 118 can be formed or placed on the skin and electrode-interconnect array. This can be done to place the multielectrode array in electrical connection with a data acquisitioning system 300. For instance, one or more electrical contacts 118 can be placed into contact with one or more interconnects 108 and then placed into contact with the data acquisitioning system 300. A conductive glue 120 can be applied to adhere the electrical contact(s) 118 to the skin.

After connected to the data acquisitioning system 300, the network of sensors (which can include a multielectrode array, for example) can be used for monitoring, measuring, and/or sensing electrical activity (voltage, a change in voltage, current, a change in current, impedance, and/or a change in impedance) of the electrode(s) 102. Neuromuscular activity of the animal can generate electrical activity in the electrode(s) 108 that is representative of the neuromuscular activity. The data acquisitioning system 300 converts the electrical activity into signals. These signals can be stored and/or further processed into data structures that are representative of the neuromuscular activity. For instance, the data structures can be transmitted to a computer device 200 having software that allow it to determine a movement, a gesture, a muscle activation, and/or a muscle relaxation based on the electrical activity. The software can measure or determine muscle response or electrical activity in response to a nerve's stimulation of a muscle, for example.

An exemplary application of these measurements can include detecting a neuromuscular abnormality. Another exemplary application can include translating the movement, the gesture, the muscle activation, and/or the muscle relaxation to an actuation signal configured to be transmitted to a prosthetic, a robotic prosthetic, or a gesture-controlled robot. For instance, the computer device 200 can cause a prosthetic, a robotic prosthetic, or a gesture-controlled robot to mimic the movement or desired movement of the animal based on the muscle response or electrical activity in response to a nerve's stimulation of a muscle. Detecting neuromuscular abnormality is only one exemplary application of the technology. Other applications can include performing electromyography, detecting/monitoring regular or irregular movement, etc.

It should be noted that the materials used for the electrically conductive ink 102, electrically insulative material 114, etc. are able to work effectively even during flexure of the skin. This is because the electrically conductive ink 102 can elastically deform when flexure occurs and generates strain on the ink 102.

### EXAMPLES

The following disclosure discusses exemplary transducers, transducer arrays, methods of producing the same, and test results.

The examples demonstrate development of a customizable and reconfigurable drawn-on-skin (DoS) MEAs capable of high-density EMG mapping from in situ fabricated electrodes with tunable configurations adapted to subject-specific muscle anatomy. The DoS MEAs show uniform electrical properties and can map EMG activity with high fidelity under skin deformation-induced motion, which stems from the unique and robust skin-electrode interface. They can be used to localize innervation zones, detect motor unit propagation, and capture EMG signals with consistent quality during large muscle movements. Reconfiguring the electrode arrangement of DoS MEAs to match and extend the coverage of the forearm flexors enables localization of the muscle activity and prevents missed information such as innervation zones. In addition, DoS MEAs customized to the specific anatomy of subjects produce highly informative data, leading to accurate finger gesture detection and prosthetic control compared with conventional technology.

The anatomical mismatch between the existing electromyographic (EMG) multielectrode arrays (MEAs) and target muscles leads to missing relevant muscle activity, highly redundant data, complicated electrode placement optimization, and inaccuracies in classification algorithms. Due to the fixed configuration of conventional MEAs, it is almost impossible to reconfigure them to match each individual's unique muscle anatomy, which is critical for physical medicine, prosthetic control, sports physiology, and rehabilitation research. This work demonstrates drawn-on-skin (DoS) MEAs as a paradigm-shifting approach to address this crucial challenge. Drawing new/erasing electrodes (without repositioning the array) allows for on-demand tunability to fully capture the spatial extent of EMG activity and improve classification. The DoS MEAs enable large-area, tunable-density, and customizable electrophysiological mapping for personalized care and treatment.

With conventional systems, the electrodes are repositioned in a trial-and-error manner to perform iterative measurements of muscle activity. The typically utilized conventional high-density MEAs are indiscriminate to the spatial arrangement of muscles with varying geometries and cannot be reconfigured in situ to the appropriate number and specific positions of electrodes to offer the most informative data, which is a significant challenge to overcome. In addition to highly redundant data/missed information, the anatomical mismatch between the existing MEAs and target muscles also results in electrode shifts and motion artifacts, further reducing the overall quality of surface EMG mapping. Devices with more deformable electrodes could potentially be useful or repurposed for reconfiguration to some extent, but they were not designed nor are readily feasible to particularly solve the anatomical mismatch issue.

Here, we present anatomically coordinated, high-density EMG mapping with customizable and reconfigurable drawn-on-skin multielectrode arrays (DoS MEAs) as the first demonstration of simultaneous EMG mapping from many direct on-skin fabricated electrodes, adapted to the muscle anatomies of multiple subjects. Such high-density DoS MEAs are achieved for the first time with substantial advancements including in situ reconfigurability of the devices, anatomical matching of the devices to the targets, high-fidelity mapping of EMG signals, and uniform and low-skin electrode impedance of many DoS sensors. Reconfigurability and anatomical matching of DoS MEAs reduces data redundancy, thus improving classification accuracy for prosthetic control. The high-density DoS MEAs are fabricated in minutes with a biocompatible conductive ink based on an Ag/poly(3,4-ethylenedioxythiophene)-poly(styrenesulfonate) (Ag-PEDOT:PSS) composite, water/acrylic emulsion-based insulator, ball pens, and stencils. The DoS MEAs show minimal variability in their electrical characteristics compared to the current wearable bioelectronics, though the drawing process is performed by a human user's hand. Comparisons of motor unit propagation mapping, innervation zone localization, and continuous EMG measurements during large muscle movements portray the higher performance of DoS MEAs relative to conventional grids, which is important in both research and future clinical contexts. DoS MEAs reconfigured to the anatomy of the wrist flexors unveil the full extent of the target muscle activity, which the conventional grid and wearable bioelectronics cannot achieve due to their fixed construction. This broadened pool of neuromuscular information from DoS MEAs that are customized to each subject's flexors and extensors provides more distinguishable data and higher accuracy myoelectric control than existing MEA technologies. Our results suggest high-density DoS MEAs as a viable customizable and reconfigurable electrophysiological recording technology for patient-specific assessments, control, rehabilitation and/or treatments. As can be appreciated from the present disclosure, the inventive techniques provide a means to obtain laboratory-quality data measurements within any setting (e.g., laboratory-quality data can be obtained anywhere and at any time, which includes outside of a laboratory setting, i.e. ambulatory monitoring).

### High-Density DoS MEA Fabrication.

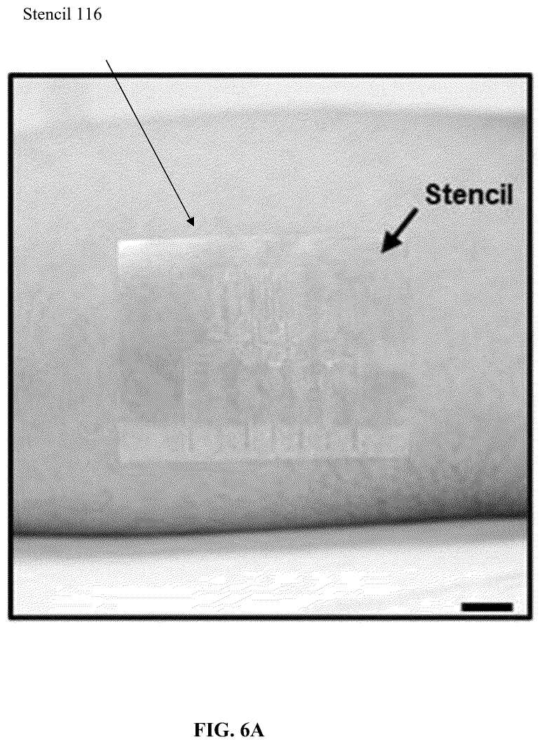

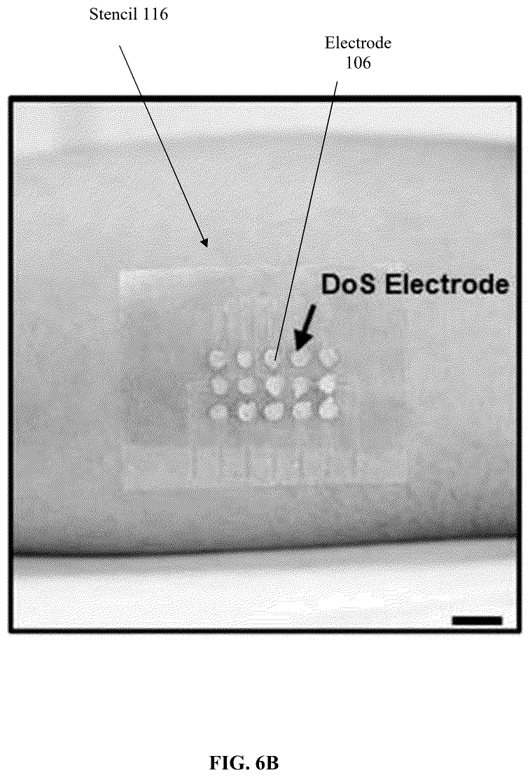

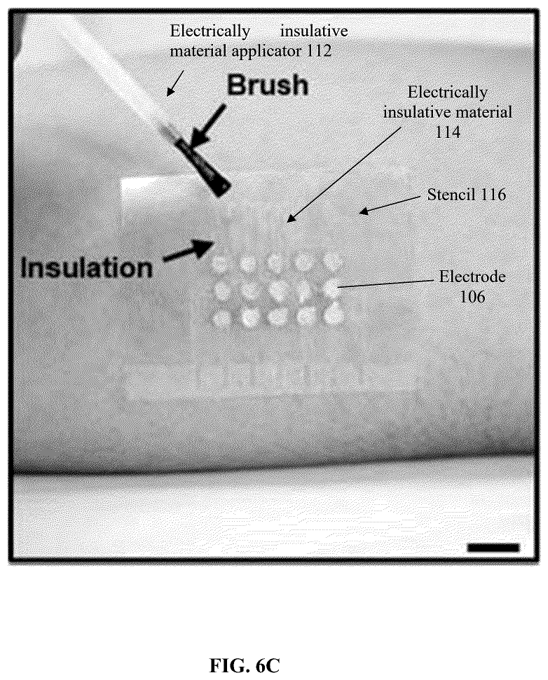

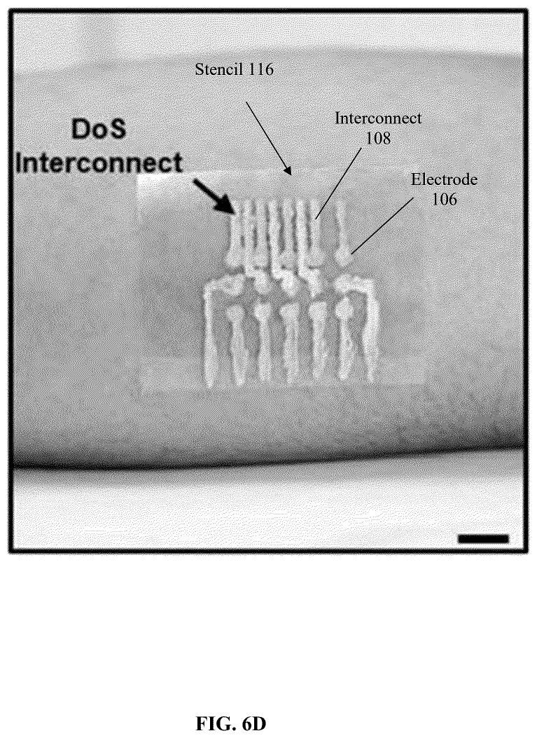

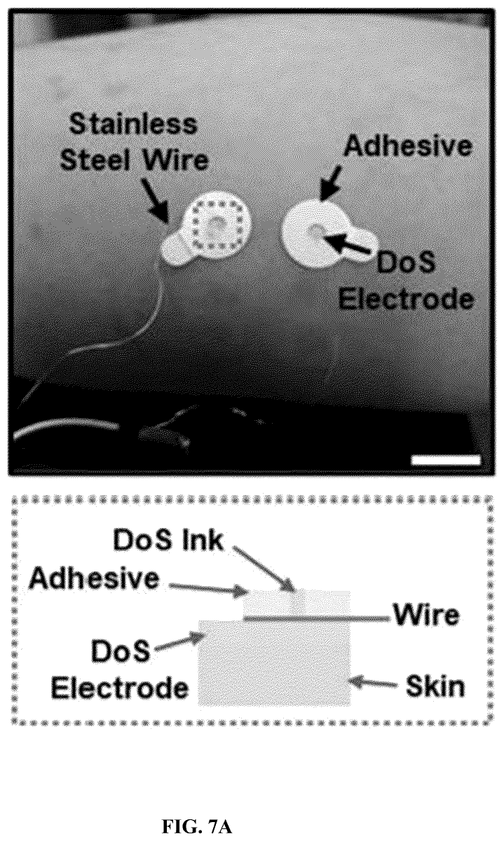

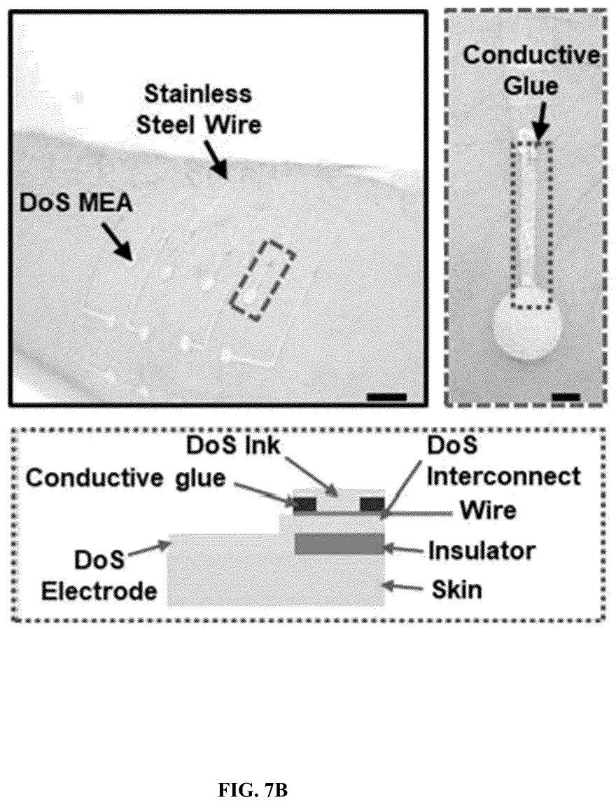

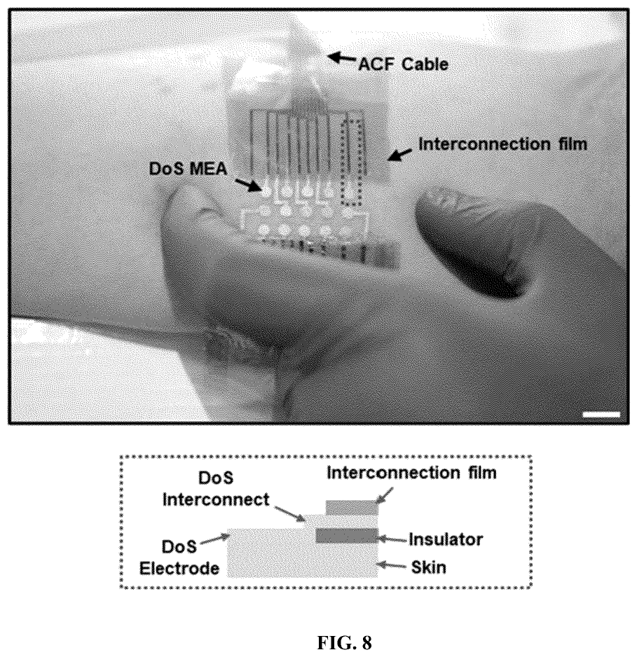

The high-density DoS MEA was prepared using a highly conductive ink, insulating material, stencils, and ballpoint pens (FIG. 1). Briefly, a stencil with the desired array configuration was prepared with a cutting machine. The Ag-PEDOT:PSS conductive ink was filled into a modified ballpoint pen, which was then used to draw into the stencil on the electrode portions or draw on the skin without a stencil. It is noted that no skin preparation is needed for the ink to adhere to the skin, since it is partially hydrophilic. We present the first anatomically coordinated mapping of muscle activity with customizable and reconfigurable high-density DoS MEAs, which is a paradigm shift of the typical iterative process used for optimizing EMG electrode placement. The DoS MEAs can be first fabricated in any desired shape of electrode arrangements, electrode sizes, low/high-densities, with/without drawn interconnects, and then altered (by erasing and drawing in new positions) based on the drawer's intuition to capture activations specific to the target muscle. This shift from the typical approach, which is indiscriminate of the muscle anatomy, ensures that the fewest number of channels are used to reveal all the relevant muscle activations from their corresponding anatomical positions, leading to low redundancy data and, thus, improved classification of hand gestures and prosthetic control, as one example. Furthermore, this approach ensures that critical information for muscle treatments, such as innervation zones and activation at the muscle belly, is not missed. It should be noted that when the DoS MEAs are fabricated with interconnects, they require an additional insulation material to avoid capturing signals from the interconnect lines. After drawing in the electrodes (see FIGS. 6A-6D) with the conductive ink, a water and acrylic emulsion-based insulating material (Pros-Aide, ADM Tronics) was brushed onto the interconnect portions of the stencil and dried at room temperature. Afterward, the interconnects were drawn with the conductive ink on top of the dried insulation. Depending on whether the interconnection is prefabricated, multiple approaches could be used to collect data from the DoS MEAs as shown in FIGS. 7A-7B and FIG. 8. The resulting array is deformable (ε≈10%) on the skin, as shown in FIG. 1C, and the ink remains functional even under 30% strain.

Furthermore, the MEAs can be scaled to muscles with areas on the order of hundreds of square centimeters, such as the trapezius (FIG. 1D) muscle. Previous reports of high-density EMG of the trapezius muscle show only partial coverage that misses information from either the upper, middle, or lower regions. The electrodes and interconnects of DoS MEAs are tuned to the subject's specific muscle shape, and the interelectrode distance is determined based on the muscle geometry and intended application. By tuning the electrode size, density, overall arrangement, and interconnect design at the point of care, the DoS MEAs were fabricated in minutes to demonstrate their adaptability to smaller muscles like the flexors and extensors of the forearm, biceps, and triceps (FIG. 1E). The arrays were even matched to the complex arrangement and shapes of some facial muscles, including the zygomaticus and risorius muscle. Although the DoS MEAs here are shown on a healthy subject, they could easily be adapted to the limb of an amputee patient, unlike the conventional planar and flexible grids. It is important to note that the dimensions of the DoS array are limited to the stencil feature size and/or pen tip diameter, depending on the usage. A minimum line width of 300 μm and line spacing of ˜200 μm were reported previously but can be improved by altering the pen tip diameter and stencil feature sizes.

### High-Density DoS MEA Impedance Characterization.

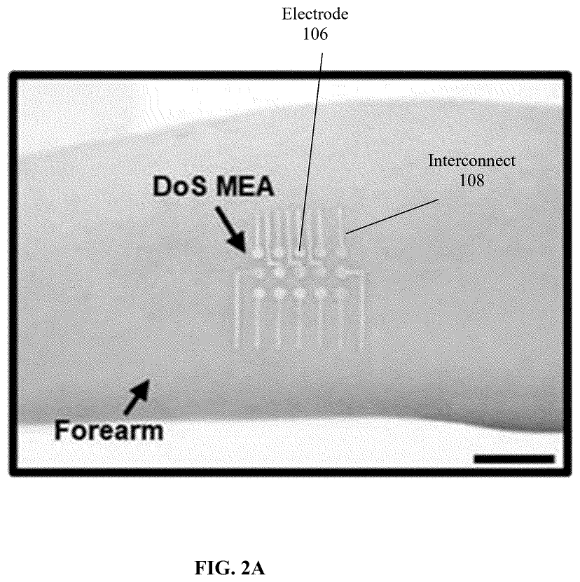

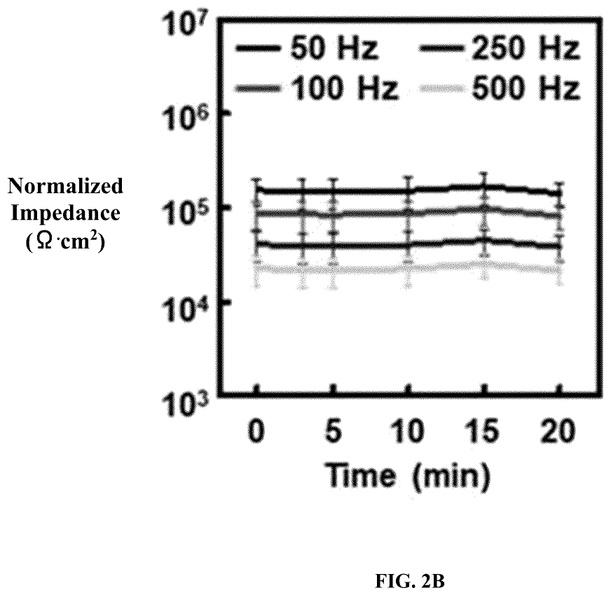

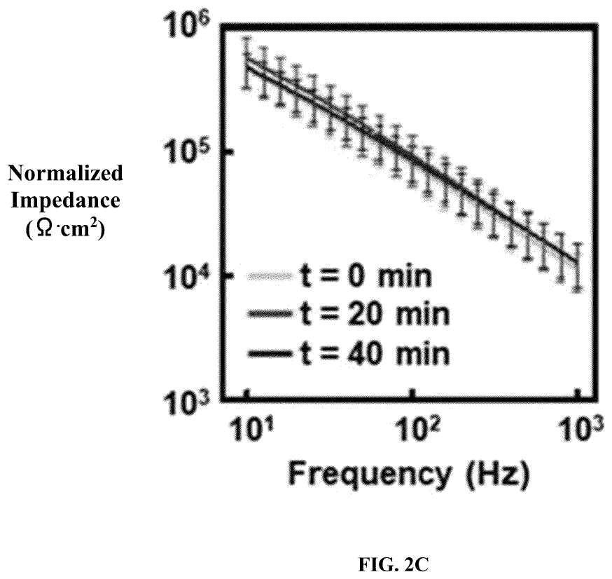

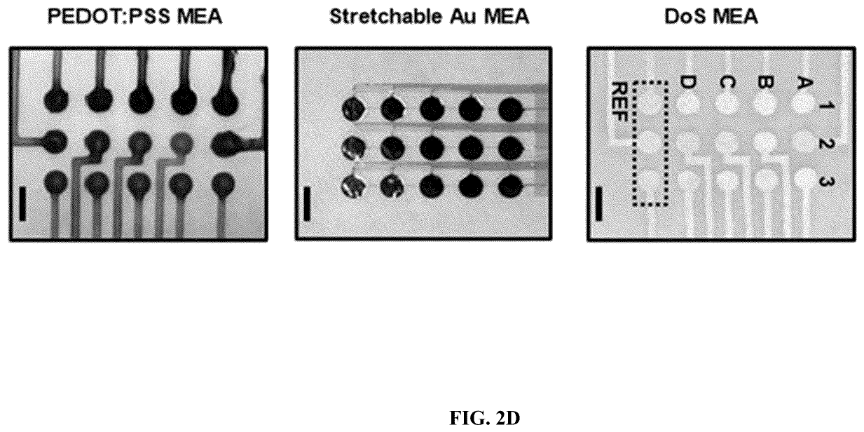

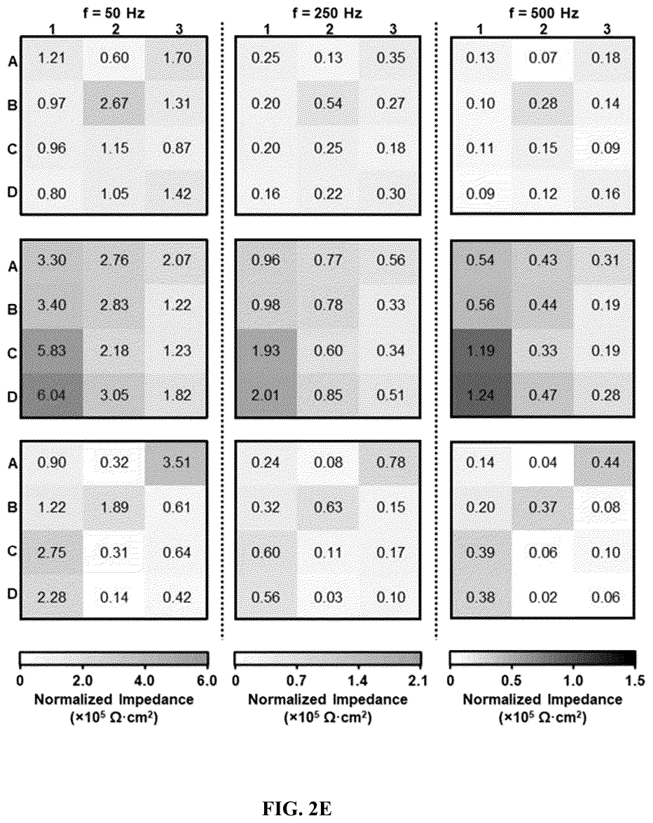

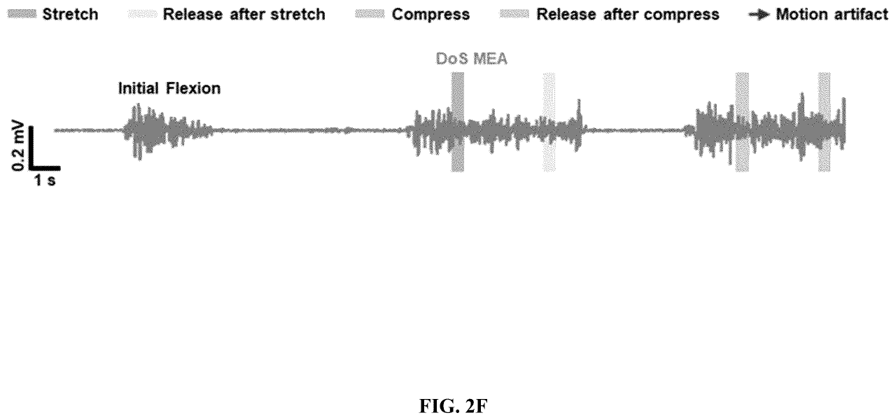

The sensing capability of the high-density DoS MEAs was validated by measuring their impedance characteristics, and they were compared with multiple types of the existing bioelectronics (stretchable Au mesh-based MEA and intrinsically stretchable PEDOT:PSS MEA) and the conventional technology, referred to herein as the Flexible Printed Circuit (FPC) grid (Twente Medical Systems TMSi, Enschede Netherlands). Each MEA was placed on the flexor muscle group of three subjects for skin-electrode impedance (SEI) measurements, with the DoS MEA being shown in FIG. 2A. The average SEI for all electrodes and electrode types from all subjects is shown for all impedance characterizations. In FIG. 2B, the normalized SEIs over time (after the entire DoS MEA appeared dry) at relevant physiological frequencies of EMG (50-500 Hz) are presented. Multiple impedance measurements every 5 min (until 20 min) after the MEAs were drawn indicate that the impedance remained stable, though the individual electrodes were drawn at different times. Furthermore, to mimic the scenario of adding additional electrodes to the DoS MEA and better accommodating the muscle shape and anatomy, we evaluated the impedance spectrum of individual DoS electrodes added to an MEA at 0, 20, and 40 minutes after the new electrodes were drawn (FIG. 2C). The difference between the impedance spectrums over time remained negligible, showing that the DoS electrodes reached a stable impedance ranging from 10⁶ to 10⁴ Ω·cm² (across frequencies between 10-1000 Hz) within minutes of being drawn as new electrodes were added to the MEA.

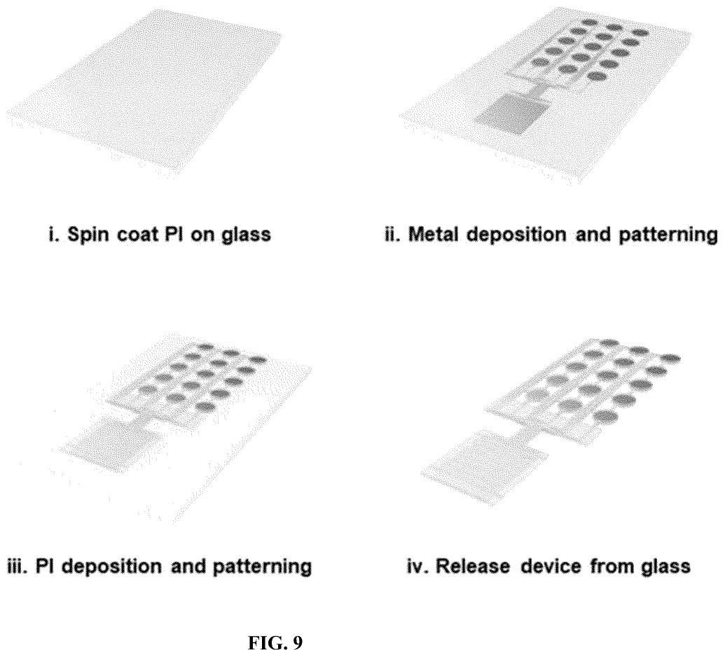

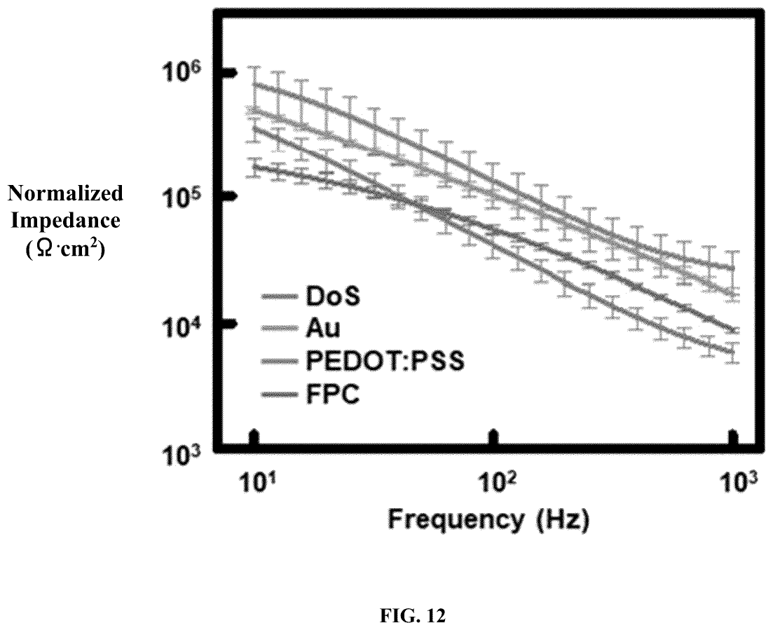

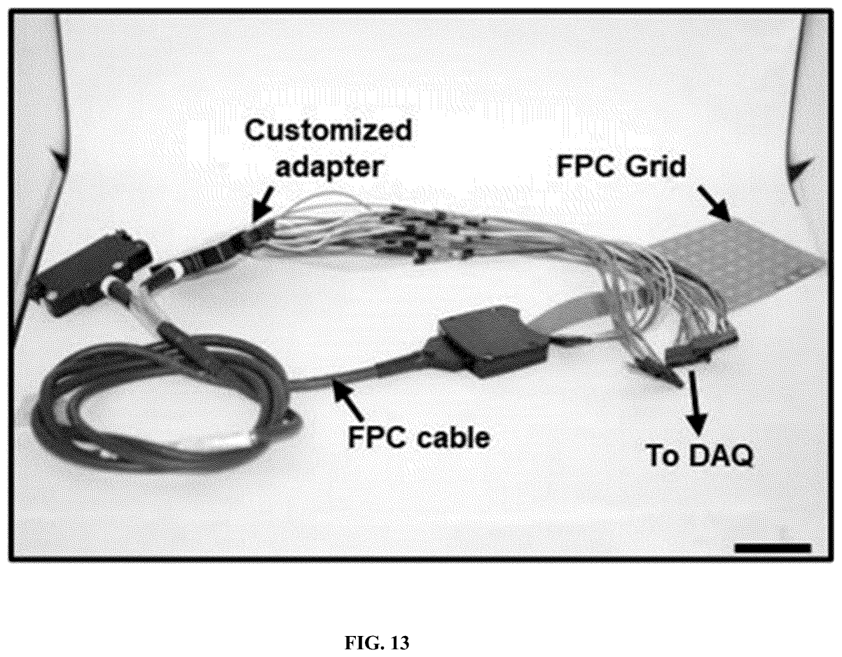

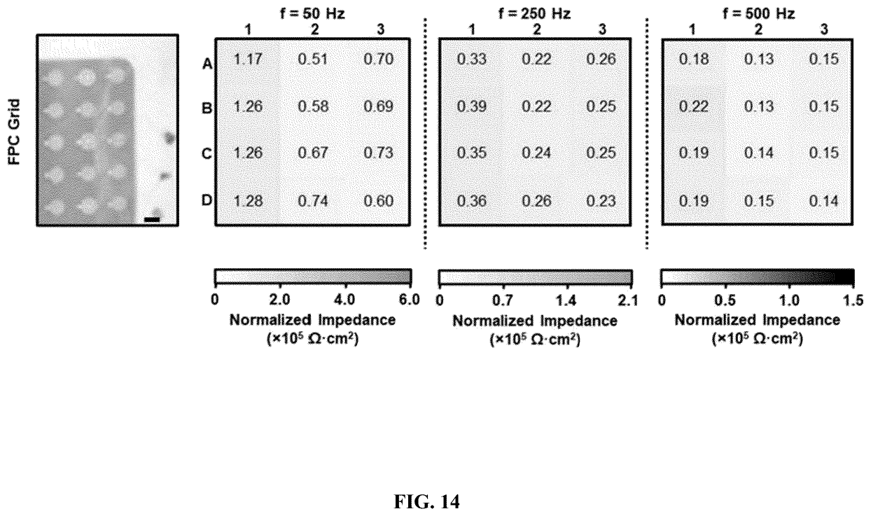

The impedance spectrums of all the technologies (fabrication and dimensions in FIGS. 9-11) were compared and revealed that the DoS electrodes show a relatively uniform normalized SEI compared to the PEDOT:PSS, Au mesh, and FPC electrodes over most of the measured range of 10-1000 Hz (FIG. 12). Average SEI heatmaps were constructed for all the MEAs (FIGS. 2D and 2E) to compare the impedance of multiple devices of each (n=3 per subject) technology for all subjects. The color uniformity of the heatmaps indicates the variance of the SEI across each of the MEAs. The heatmaps show minimal variation among all the electrodes in the DoS MEA, even though each electrode is drawn with a slightly different drawing speed and has a relatively more varying thickness, unlike the other electrode types. It is important to note that although this physical difference exists between the individual DoS electrodes and other electrode types, the difference is not sufficient to greatly affect the SEI. The impedance of the DoS electrodes is negligible (order of Ω) compared to the SEI (order of MO). In addition, it should be noted that although there are variations among the electrodes for the PEDOT:PSS and Au mesh-based MEAs, overall, they still show similar and higher SEIs, respectively, compared to those of the DoS MEA. The FPC grid (required custom data acquisition—FIG. 13) shows both high uniformity and lower SEI (FIG. 14), expected of a gel-based conventional technology. However, at the higher frequencies of EMG (>250 Hz), the electrodes of the DoS MEA show lower impedance than those of the FPC grid and other technologies. These results corroborate the use of DoS MEAs for electrophysiological signal mapping and suggest that adding electrodes to a customized DoS MEA can be done rapidly while maintaining relatively uniform impedance characteristics.

### EMG Signal Quality During Skin Deformation-Induced Motion.

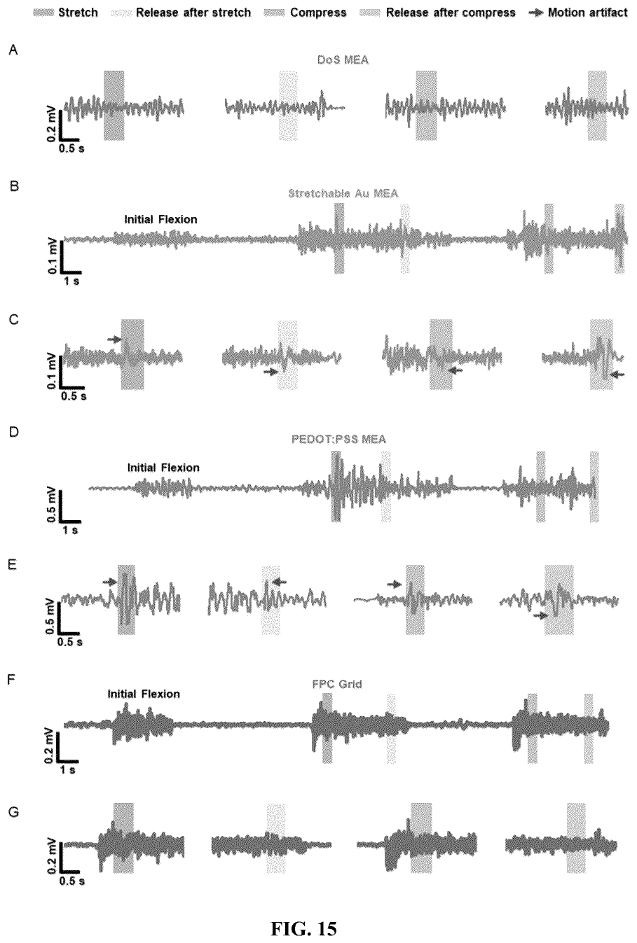

Movement artifacts are a substantial issue in EMG sensing as noise captured from the motion can overlap with the low-frequency content comprising true muscle activity. A further issue particularly attributed to measuring EMG signals is that certain muscles shift underneath the skin and are at different positions relative to the electrodes, depending on the level of muscle activation and body posture. To evaluate the effect of relative movement between the skin and underlying muscle, a relatively stationary group of muscles (finger and wrist flexors) was chosen to ensure the muscle stayed in place while the skin was deformed. This approach ensured that minimal muscle movement relative to the skin occurred so that the artifacts could be identified as low-frequency changes to the baseline of the EMG signal during contraction, attributed solely to the skin deformation. Representative EMG signals averaged across each MEA from a single subject are shown in FIG. 15. The EMG signals recorded with the DoS MEA show no artifacts during the stretching, compressing, or releasing motions (highlighted portions) while the subject flexes (FIG. 2F), zoomed-in view in FIG. 15). However, the Au and PEDOT:PSS MEAs show substantial artifacts (red arrows), as shown in FIG. 15. The artifacts are clear deviations of the baseline and are relatively slower oscillations that could be removed with a high pass filter with a cutoff above 20 Hz.

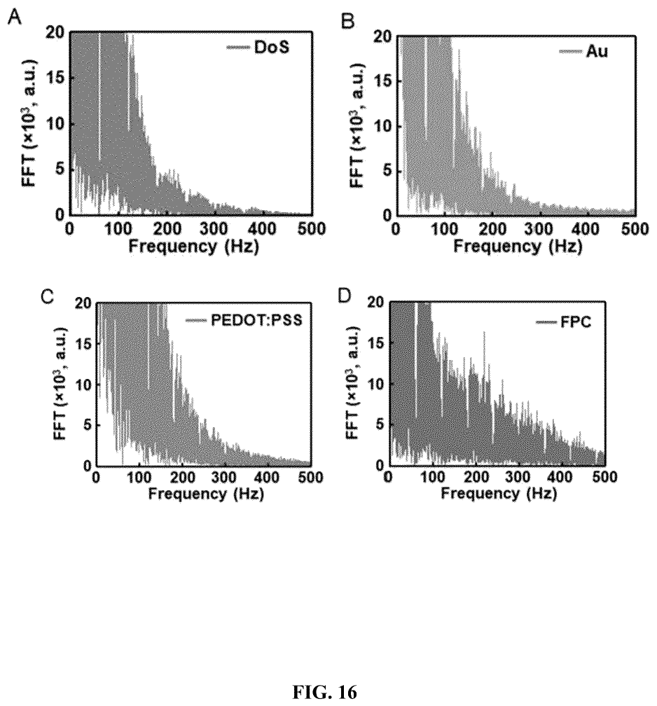

It should be noted that although the strain distribution across the MEAs during the induced motion may not have been uniform across the array during the skin deformations, the effect of movement induced by skin deformation is clearly distributed throughout the electrodes in the Au and PEDOT:PSS MEAs to some extent, as evidenced by the averaged EMG signals. Furthermore, the same experiment was conducted using a 3×8 portion of the FPC grid, which showed no artifacts during any deformation (FIG. 15); this can be due to the strong adhesive. It is important to consider that the extra adhesive may only offer temporary benefits: repeated movement underneath the adhesive could cause delamination of the grid in the long term. Nevertheless, considering the longer-term wearability of DoS electronics, motion artifact-less data, and high-fidelity EMG data (FIG. 16), DoS MEAs are potentially well-suited for the dynamic, real-world ambulatory measurements.

### High-Density DoS MEAs for Muscle Activity Assessments.

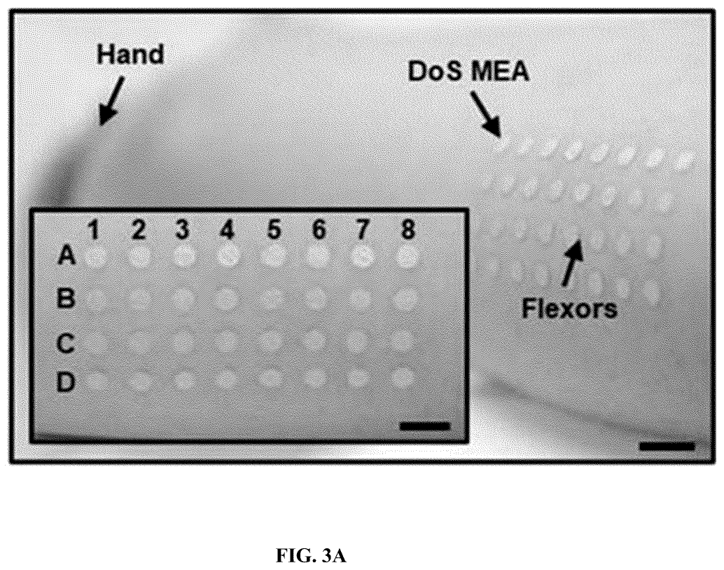

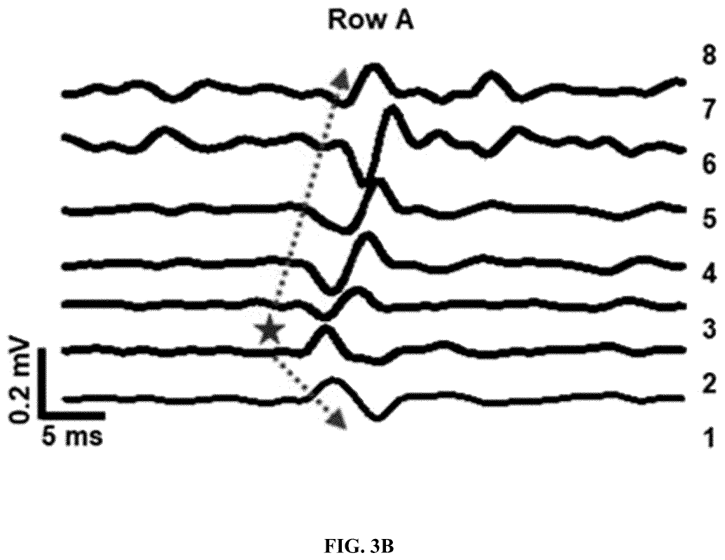

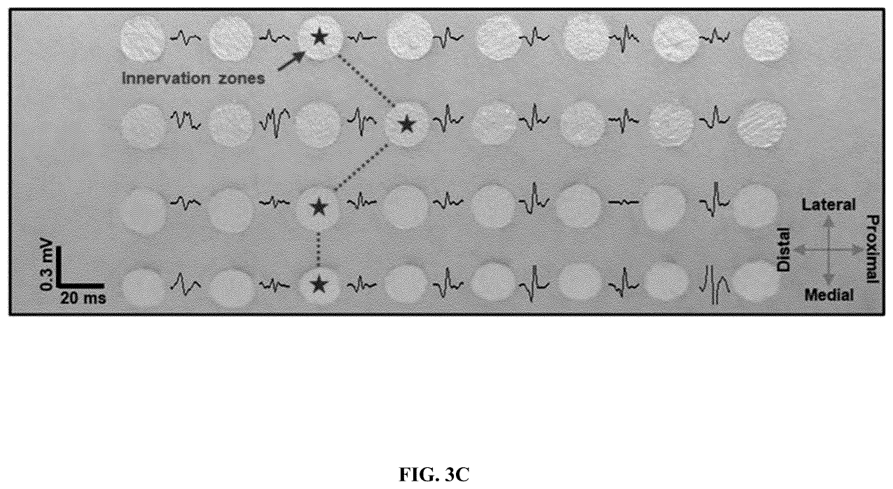

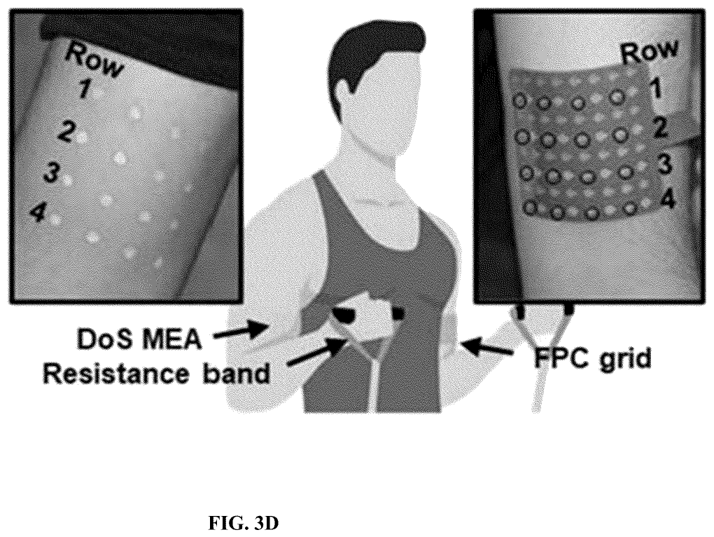

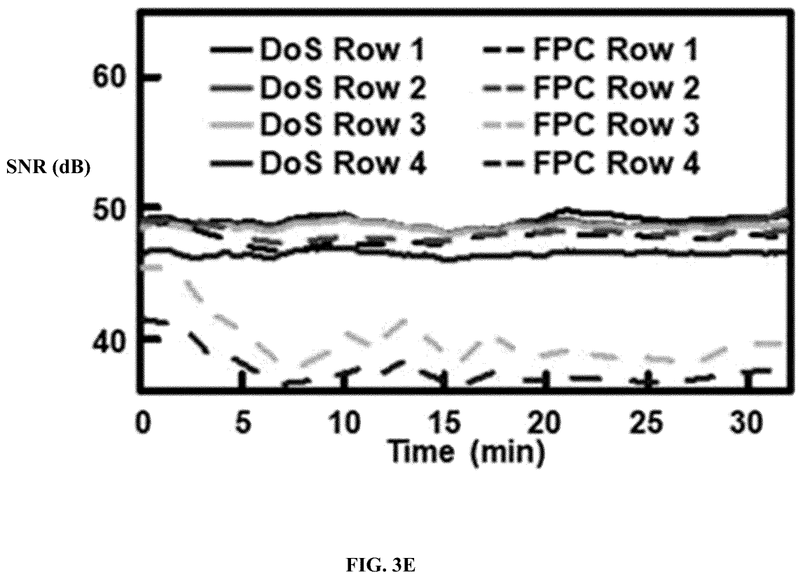

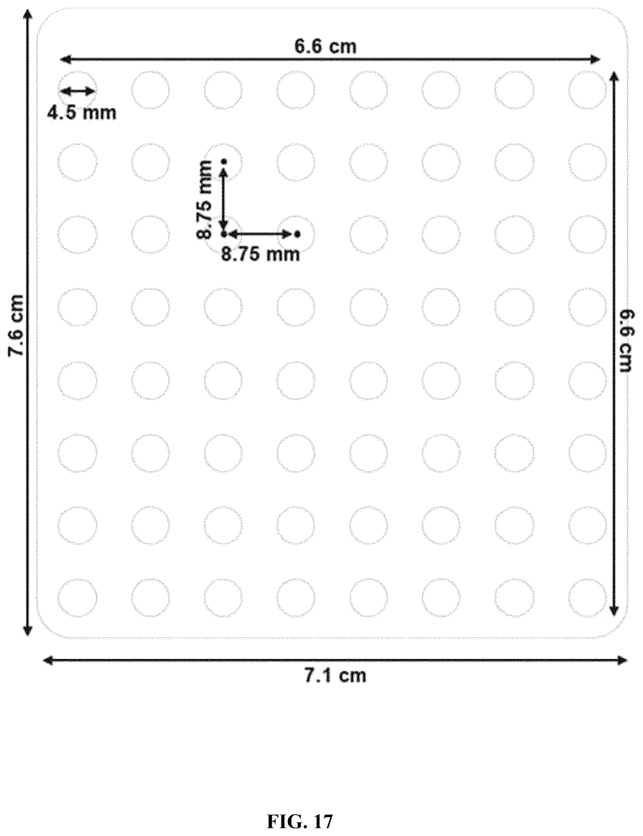

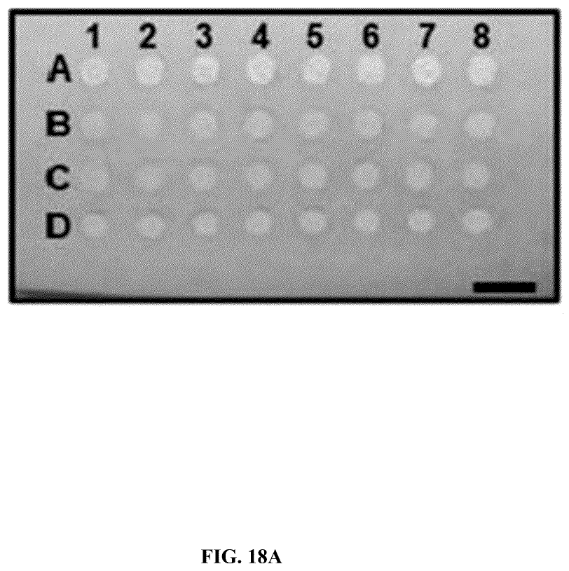

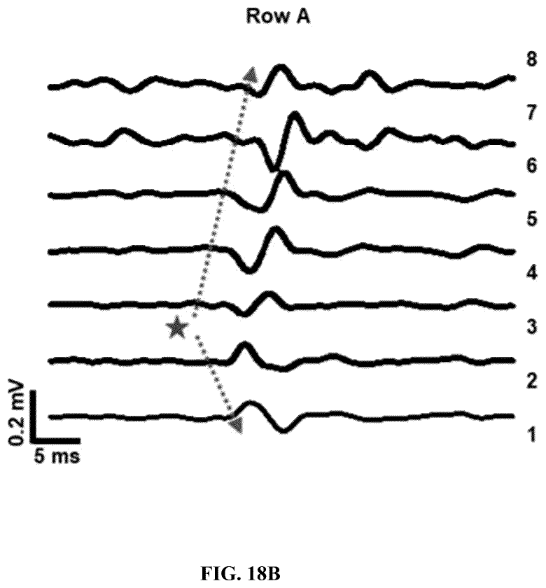

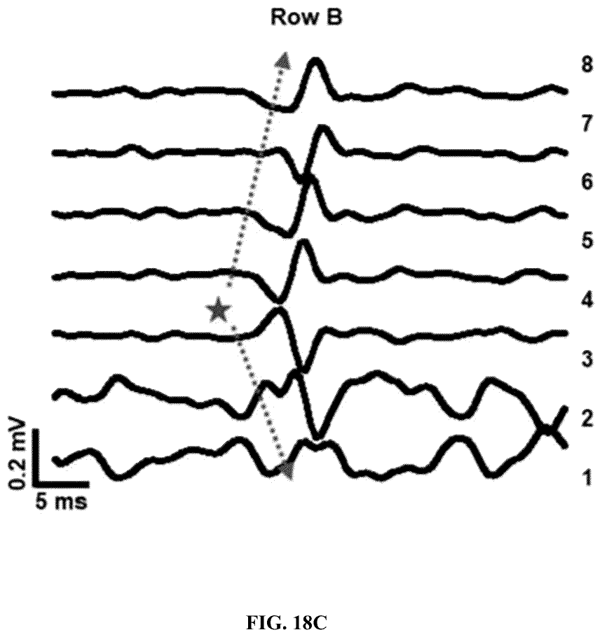

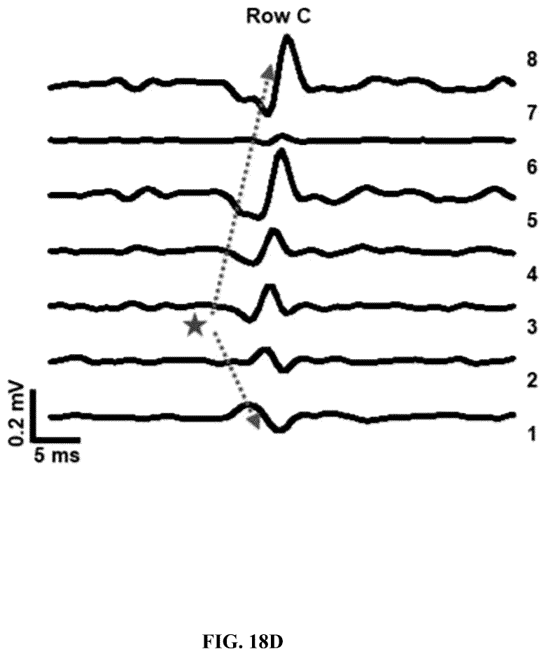

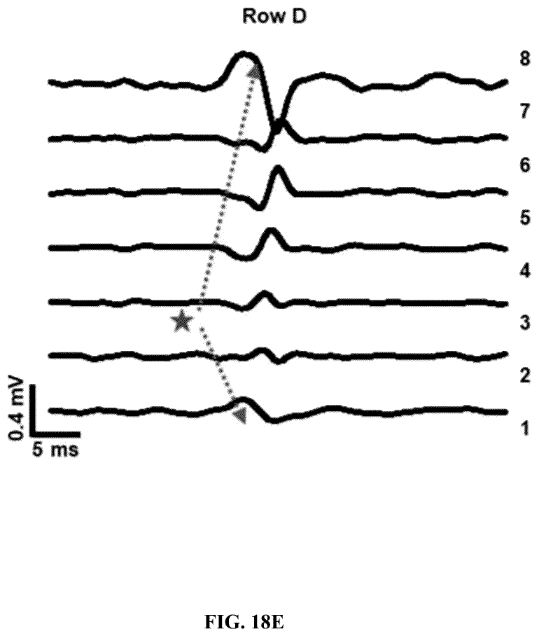

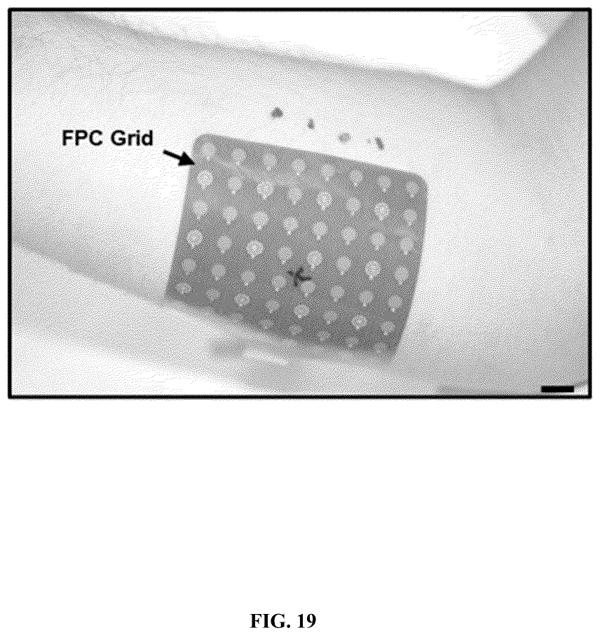

Capturing the activity at the sites where nerve terminal branches synapse with muscle fibers, e.g., innervation zones (IZs), can help improve the understanding of the muscle activity and morphology in normal and pathophysiological conditions. One application of high-density EMG is to localize the IZs of muscles as potential therapeutic targets to treat movement disorders, dystonia, and spasticity. The IZs are located through the study of motor unit action potential (MUAP) propagation. The DoS MEAs can be tuned to have varied electrode densities (low to high) and capture motor unit activity when fabricated in high-density formats. High-density MEAs usually have >32 channels, <5 mm electrode diameter, and <10 mm interelectrode spacing. The DoS MEA, configured in a high-density format matching the dimensions of the commercial FPC electrode (FIG. 17), was placed on the wrist flexors (FIG. 3A) of three subjects again. Representative results are shown in FIG. 3B. The propagation results from row A (FIG. 3B) indicate a possible innervation zone among the lower channels, oriented closer to the wrist (FIG. 3C). The other rows (B, C, and D) of the array show similar propagation maps (FIGS. 18A-18E), which are in accord with row A in terms of the spatial locations of the possible innervation zones. The localization of all IZs from each row of the grid reveals an IZ band, representing the collective sites of motor unit innervation. From these high-density mapping results obtained with the DoS MEA, the average muscle fiber propagation velocity was calculated to be 6.33 m/s and individual motor units were detected by the DoS MEA. As a comparison, the FPC grid was also placed in the same location (FIG. 19) to detect motor units. The results obtained with the high-density DoS MEA are promising for therapeutic applications in muscle recovery and prosthetics.

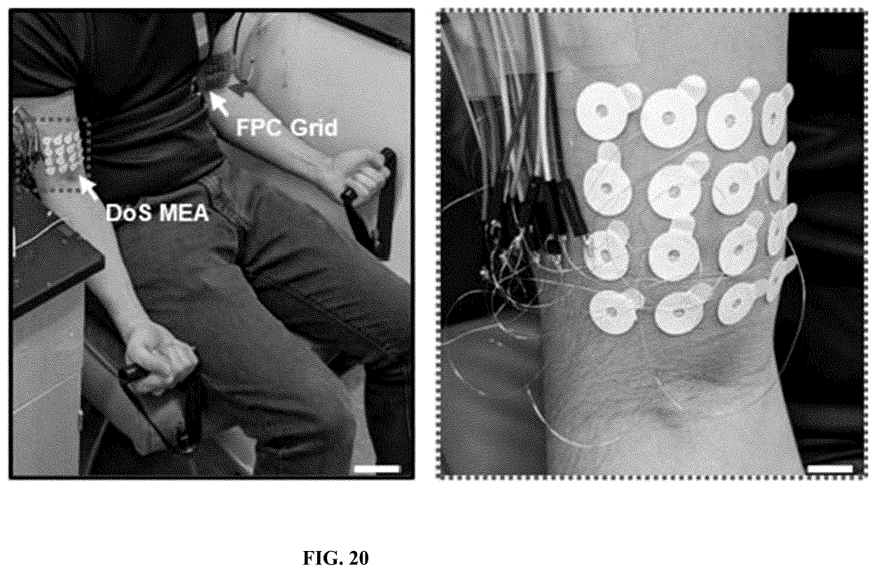

In another comparison of the DoS MEA (in a low-density format) and FPC grid in a manual muscle test, we evaluated the quality of the EMG signal over time during substantial movement of the biceps brachii muscle (FIG. 3D). Such large muscle movements under the skin are particularly relevant to exercise and sports physiology. Although the adhesive of the FPC grid is quite strong upon application, over just a few repetitions of the bicep contraction during seated dynamic bicep curls with a resistance band, a portion of the grid nearer to the crook of the elbow delaminated (FIG. 20). An image of the setup is also provided in FIG. 20. The averaged EMG signals (per row) from the lower rows (FPC grid Row 3 and 4) showed an overall lower SNR (e.g., <45 dB) compared to those of the top rows of the grid and all the rows of the DoS MEA as plotted in FIG. 3E. In applications where large muscle movements cause substantial deformation at the skin surface, such as the above manual muscle test, the DoS MEAs appear as a viable alternative to the conventional grids.

Customizing the electrodes to the muscle anatomy can offer the appropriate resolution and better classification accuracy from pattern recognition algorithms without creating redundancies. Redundancies in EMG data are interference signals that decrease the differentiability of the data for classification. All of the current wearable bioelectronics and conventional technologies used for surface EMG are fixed and indiscriminate in their construction. Considering that most prosthetics are fitted based on the underlying remaining muscle activity and that those activities are detected by placing and repositioning electrodes using a trial-and-error approach, DoS electronics exclusively enables the development of reconfigurable MEAs to map all relevant spatial information at the point of care. It should be noted that electrode shifts, which occur during repositioning of the prosthetic socket relative to the electrodes, could also be avoided with DoS MEAs as they remain in position when the sockets are donned/doffed. As an example of customizing and reconfiguring DoS MEAs, we iteratively altered the arrangement of DoS electrodes relative to the commercially available FPC grid and analyzed the spatial features of each arrangement. The FPC grid used here served both as a reference to fix the position of the DoS electrodes and as an example of an indiscriminate, prefabricated device. It should be noted that the FPC grid would not be necessary in practice, and it is only used here for demonstration. Changing from one arrangement to another (from arrangement 1 to 3) meant that the misplaced DoS electrodes were erased (using a wet cotton swab or paper towel), and new electrodes were drawn into the positions for the next arrangement. The wires for the new positions of electrodes (depending on whether interconnection lines were drawn) could easily be attached. For example, after fabricating the new electrodes, interconnection lines could be drawn without a stencil and subsequently have wires attached on top or wires could be directly attached onto the new electrodes without needing to create an entirely new MEA.

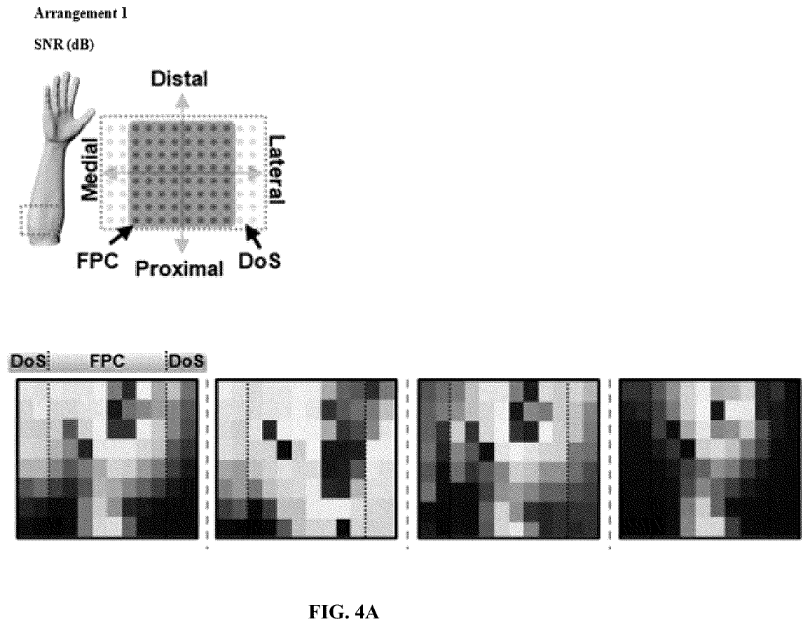

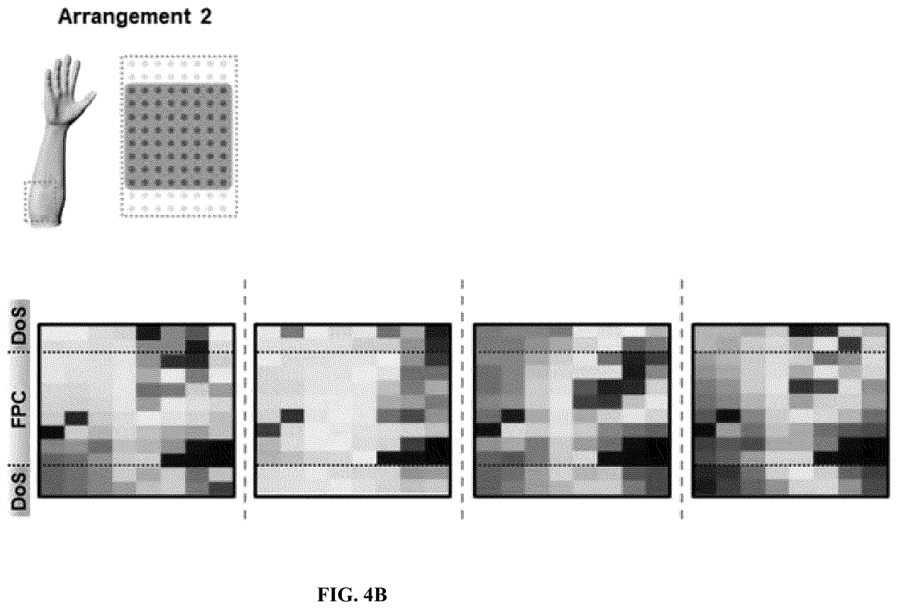

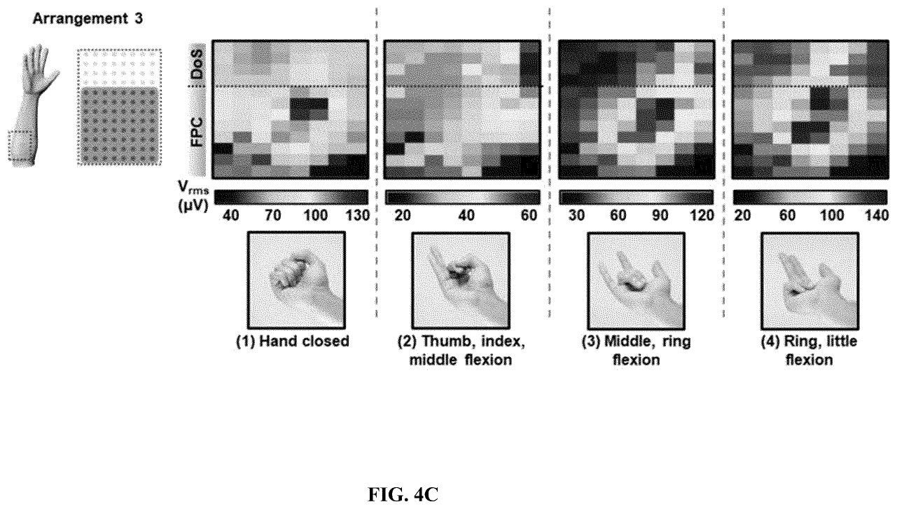

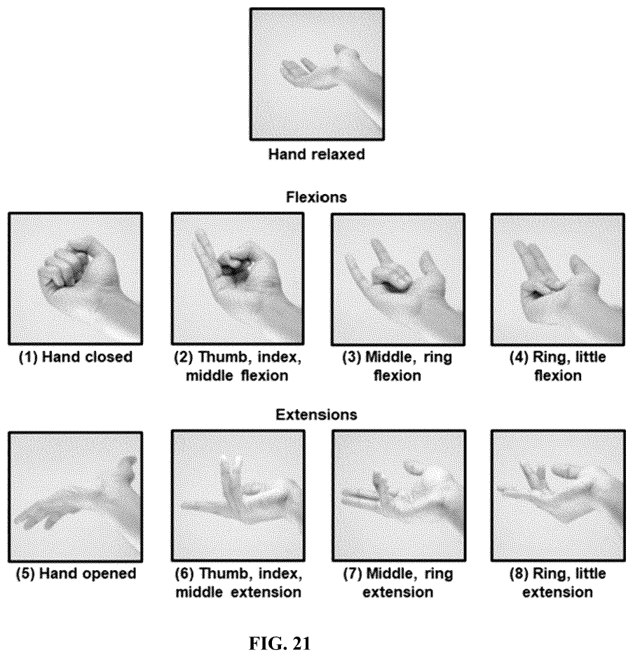

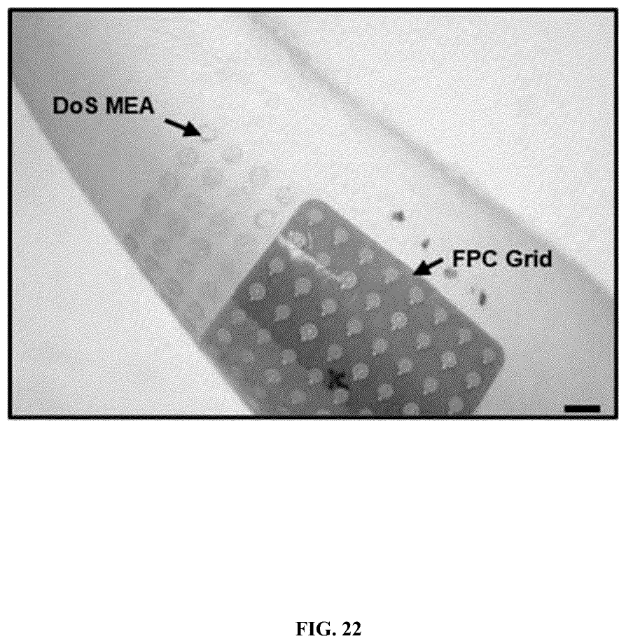

In FIG. 4A, the FPC grid position was fixed on the wrist flexors while the DoS MEAs were drawn in different spatial positions to determine the extent of the muscle excitation during different finger flexions. Those included (1) hand closed; (2) thumb, index, middle flexion; (3) middle, ring flexion; and (4) ring, little flexion. For reference, a subject's hand in a relaxed state is shown in FIG. 21. In FIG. 4A, the DoS electrodes were arranged in 8×2 arrays beside the FPC grid to improve circumferential coverage of the forearm. The DoS electrodes increase the number of channels and spatial area, supplementing the FPC grid. The heatmaps have vertical dashed lines, which indicate the edges of the activity recorded from the FPC grid. In this arrangement (arrangement 1), the EMG activities recorded from the DoS electrodes are to the left and right of the dashed lines on either side of the heatmaps in FIG. 4A. For gestures (1), (3), and (4), the voltage maps show a central pattern of activity that is more distal than proximal to the body in the upper portion of the map. The heatmap for gesture (2) shows activity that extends in the proximal direction, and the additional rows of DoS electrodes reveal activity in the lateral direction, which the FPC grid misses. The DoS electrodes consistently reveal additional regions of muscle activity for this particular gesture, even when the DoS array is reconfigured in arrangement 2 (FIG. 4B) and arrangement 3 (FIG. 4C). However, all the maps in FIG. 4A do not show discernable edges of the muscle activity, with potentially missed activity that is more distal/proximal relative to the mapped area.

In another attempt to better localize the center of muscle activity (FIG. 4B), the DoS electrodes were drawn along the length of the forearm in 2×8 array formats on either side of the FPC grid. The heatmaps reveal more information due to the DoS electrodes being more distal than in the previous arrangement. The horizontal dashed lines indicate the edges of the FPC grid in these maps (FIG. 4A). Above and below the dashed lines represent the activity recorded from the DoS MEAs. Still, arrangement 2 falls short in that there continues to be more activity along the length of the forearm, even more distal to the current electrode layout. The heatmaps in FIG. 4C finally constrain the activity of gestures (1) and (4), with the DoS electrodes being reconfigured to be entirely on one side (more distal) of the FPC grid. In the heatmaps in FIG. 4C, all the data above the dashed line is from the DoS MEA. Arrangement 3 has the best configuration of DoS electrodes compared with the other arrangements since the center of activity can be more clearly identified after tuning the electrodes to better positions. Still, it could be further improved with more rearranging of the DoS electrode positions to define the full spatial extent of the activity for gestures (2) and (3). It should be noted that although the DoS MEAs are configured in rectangular array layouts in this example, determining the full spatial extent may not require uniformly arranged layouts of electrodes and instead could require arbitrarily shaped MEAs, which cannot otherwise be achieved on demand by prefabricated grids after being placed on the skin. Through these various arrangements, the reconfigurability of DoS MEAs illustrates the ease of revealing further spatial information, which could be used to better evaluate the function of muscles in both healthy and amputee patients without greatly increasing the redundancy of the data. This approach also enables iterative localization of the center of activity in the activation maps, which could be used as highly informative image inputs to convolutional neural networks for gesture classification. Furthermore, it is important to acknowledge that although individual sEMG electrodes or the use of additional grids may offer some level of customizability or improved areal coverage, they do not simultaneously offer the benefits of DoS MEAs, including movement artifact-less recording, gel-free and imperceptible wearing, simple/inexpensive fabrication, deformability, ultra-conformal contact, and long-term usage all while minimizing the redundancy in EMG data and reducing the number of channels. In addition, this demonstration highlights the potential cooperative use of DoS MEAs with existing MEAs as a step toward more personalized medical sensing.

### Customized DoS MEAs for Finger Gesture Classification and Prosthetic Hand Control.

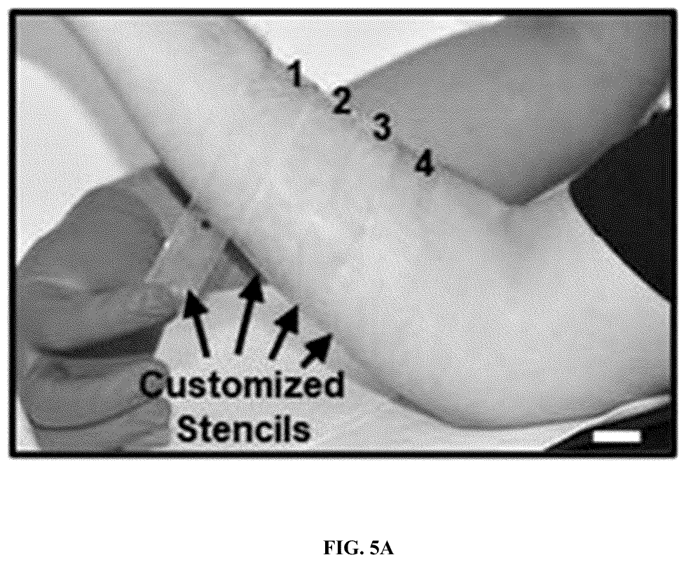

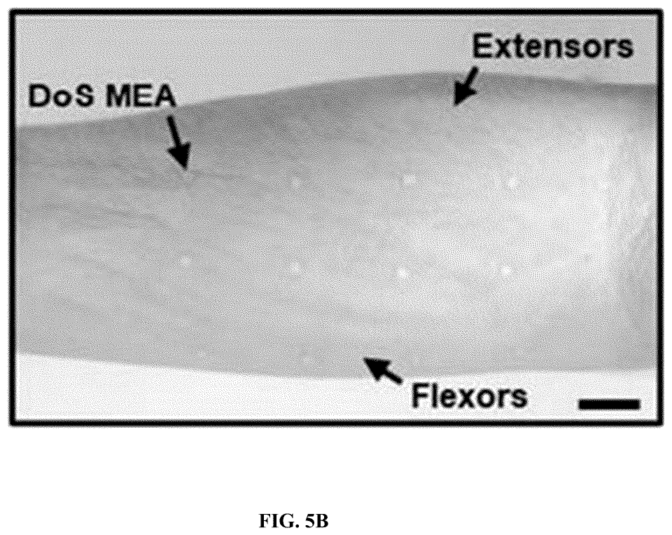

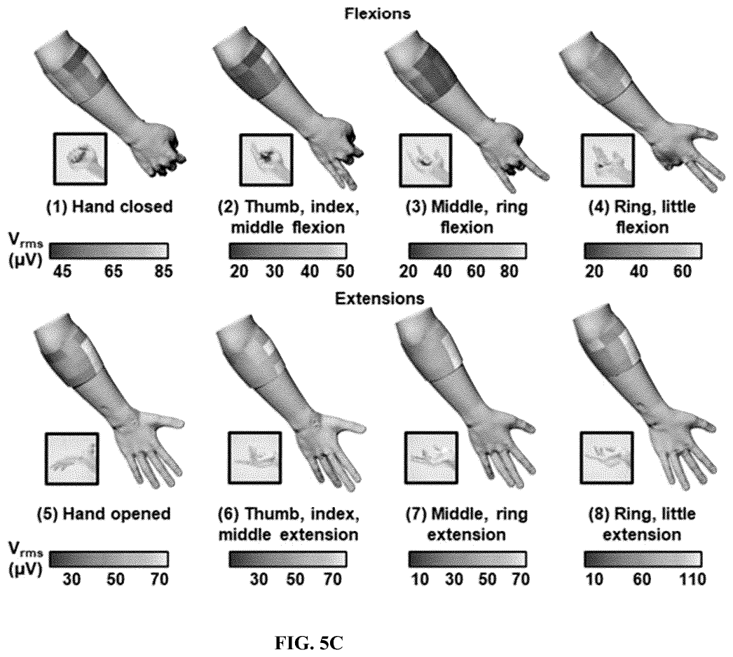

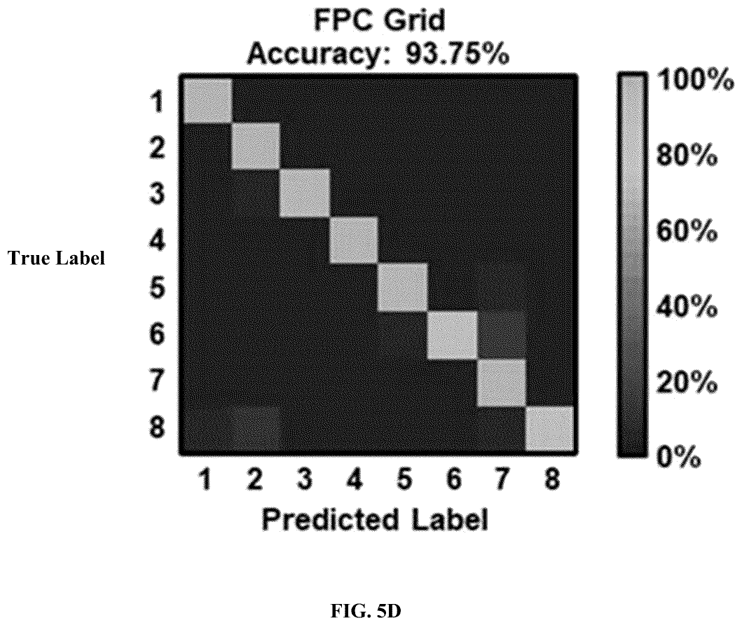

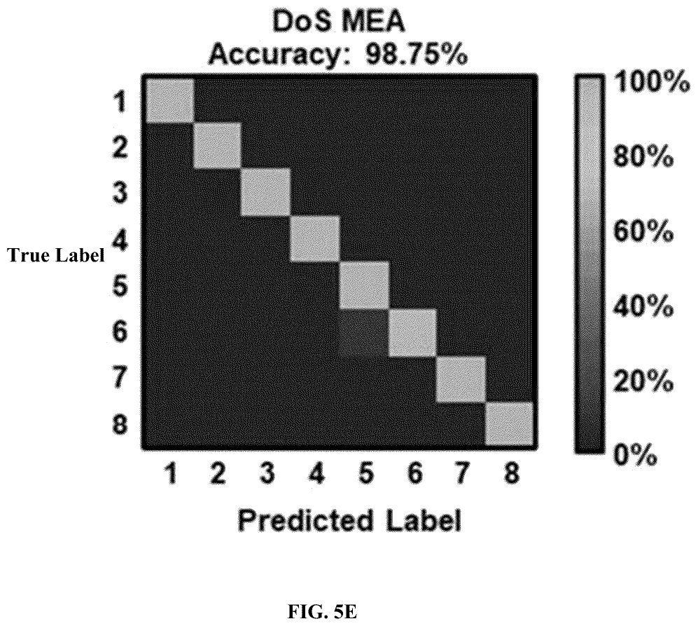

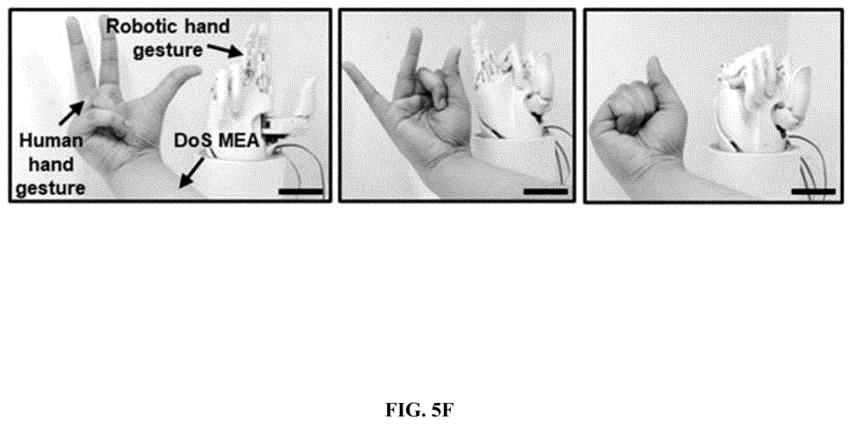

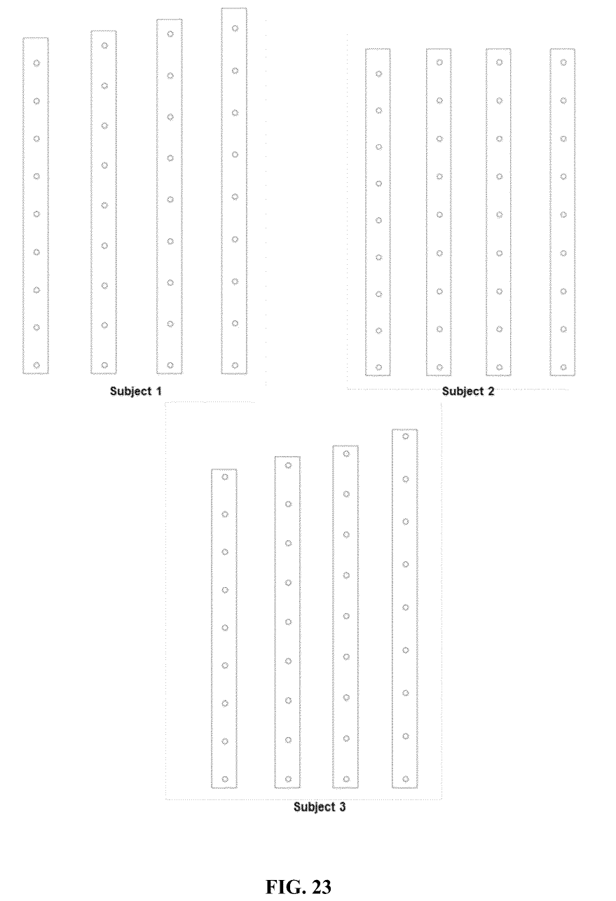

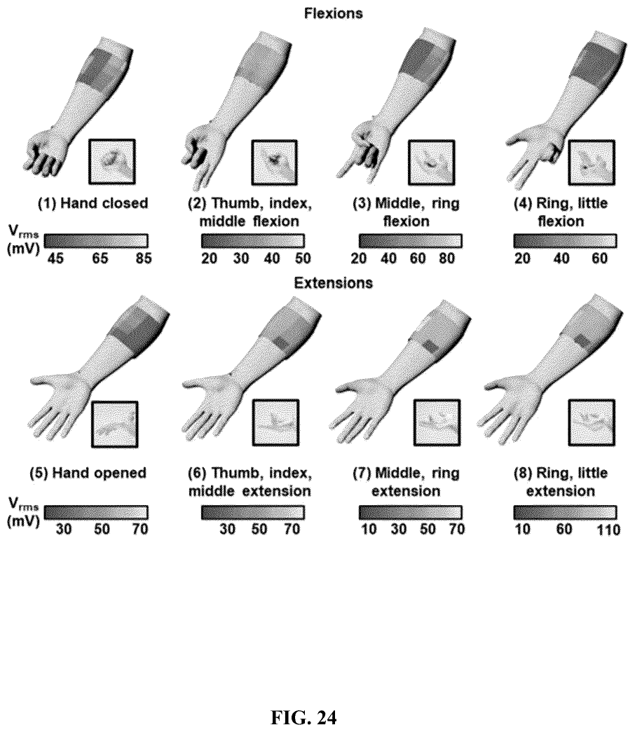

Each individual's unique anatomy calls for customizable and reconfigurable sensing platforms for accurate, personalized care. Various studies demonstrate the importance of EMG arrays customized to the anatomy of the target muscles with varying electrode dimensions, spacing, and overall sizes. The customizability and reconfigurability of the DoS MEAs reduce data redundancy and improve classification accuracy for prosthetic control, distinguishing this work from the existing studies, all of which do not demonstrate reconfigurability. The completed DoS MEAs made with customized stencils (FIG. 5A and FIG. 23) covered both the wrist/finger flexors and extensors, as shown in FIG. 5B. Each subject was asked to perform the four aforementioned gestures along with the following extensions, including (5) hand open; (6) thumb, index, middle extension; (7) middle, ring extension; and (8) ring, little extension. Lateral views of the forearm show that across all flexions (gestures 1-4), the more proximal and posterior portion of the forearm shows relatively higher excitation (FIG. 5C). In addition, gestures (1), (2), and (4) show some excitation over the extensors, which is in agreement with reported literature. For all the extension gestures, the lateral views of the forearm show consistent excitation across the group of extensors (FIG. 5C). The medial views of the forearm for both flexion and extension gestures are shown in (FIG. 24). Compared to the excitation maps of the lateral view, those of the medial view show comparably less activity for both sets of gestures.

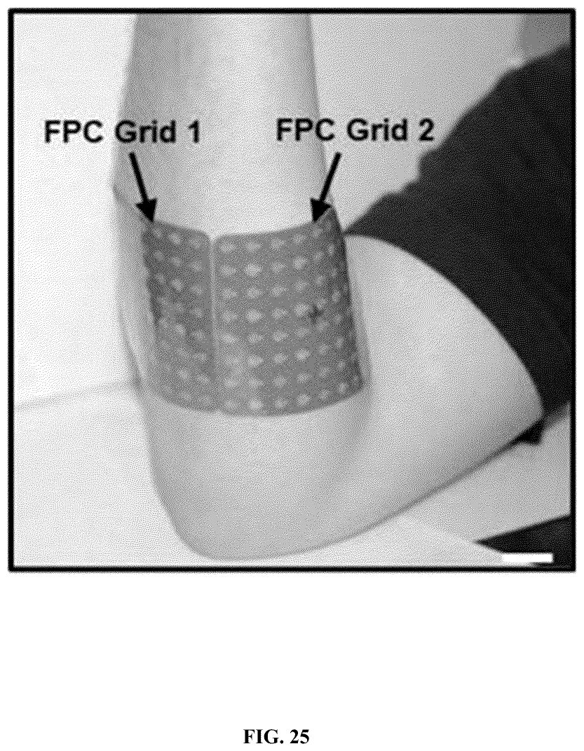

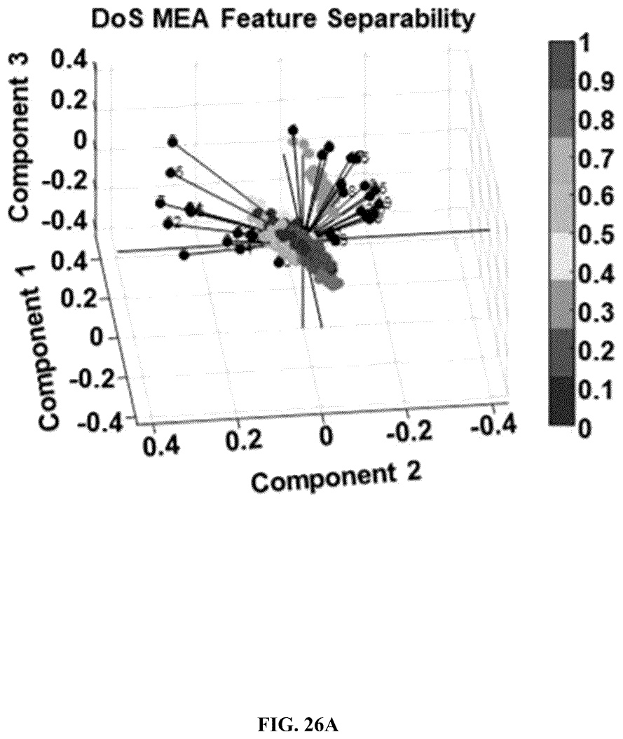

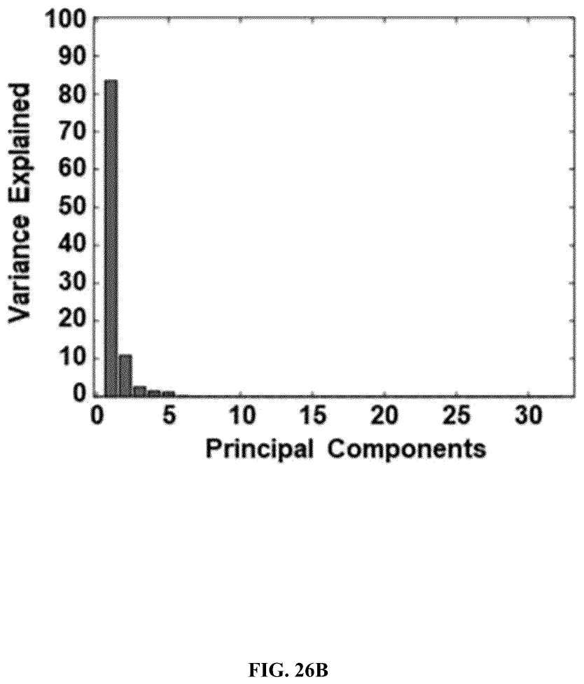

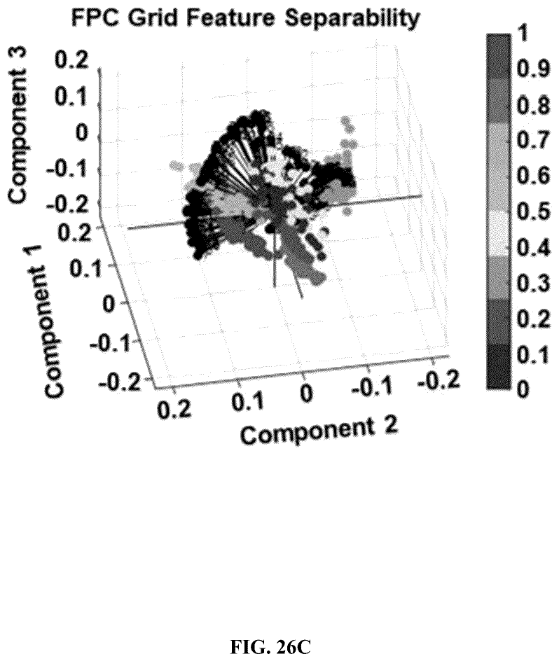

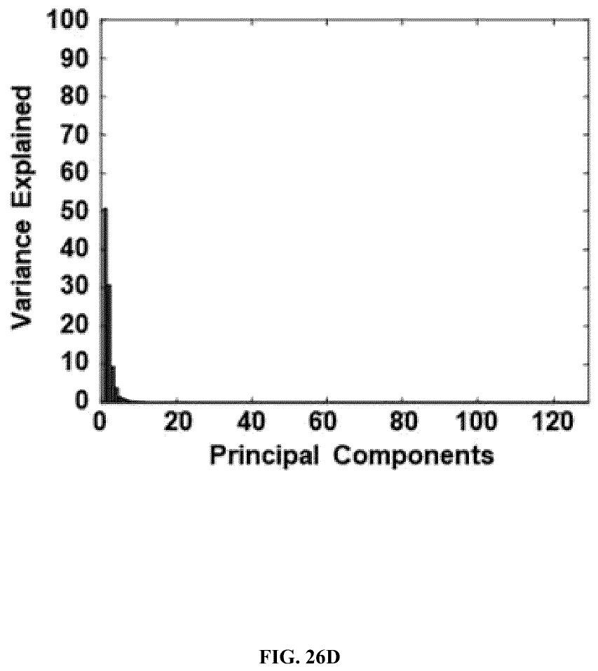

Controlling prosthetic hands with surface EMG is a promising strategy to improve the quality of life for patients with impaired mobility of limbs. We compared using the custom DoS MEAs with two FPC grids placed next to each other (FIG. 25) for controlling a prosthetic hand based on the different finger gestures. Here, the Vᵣₘₛ features extracted from the EMG data were fed into a linear discriminant analysis (LDA) classifier, both of which are among the simplest features and pattern recognition algorithms to utilize, respectively. Offline analysis of the EMG data (FIGS. 5D and 5E) showed higher classification accuracy obtained with DoS MEAs (98.75%) as compared to the FPC grids (93.75%). It should also be noted that this was achieved using only 32 DoS electrodes compared to the 128 FPC electrodes. This difference is likely due to the redundancy of the data and, therefore, lower Vᵣₘₛ feature separability as indicated by the principal component analysis results shown in FIGS. 26A-26D.

Additionally, due to their placement, the FPC grids could not obtain the same spatial information as the DoS MEA, and additional grids would be necessary, further complicating acquisition and postprocessing. With an online classifier and the customized DoS MEA, the subjects were able to control a prosthetic hand in near real-time, as shown in FIG. 5F. Taken together, the surface EMG and classification results from the customized DoS MEAs are promising for use in both healthy and patients with disabilities for accurate prosthetic control.

### Discussion

The DoS MEAs presented in this work are the first demonstration of high-density electrophysiological signal mapping with devices fabricated in situ. The approaches for customizing the DoS MEAs, collecting data from them, and reconfiguring them to obtain the highly informative EMG data indicate a feasible practice that could be performed by anyone that has a general understanding of human muscle anatomy. In addition, future computer-aided simulation and design of the geometries of the DoS MEAs could provide improved performance. On top of overcoming the limitations of high redundancy in EMG data and fixed construction of the existing MEAs, DoS MEAs bring several advantages, including relatively uniform impedance characteristics regardless of the manual drawing process, motion-artifact less EMG data in the presence of skin-deformation-induced motion, and detection of critical neuromuscular properties in both high- and low-density formats with high-fidelity EMG signals. Importantly, the ability to customize the DoS MEAs and reconfigure them is a method that most naturally suits the iterative manner by which the optimal positions of EMG electrodes are typically determined. Although the drawing process is completed with a stencil, purely hand drawing without a stencil is also possible particularly when the device geometry is not critical to its performances. Other drawing methods, such as contoured 3D printing, could also be feasible. DoS MEAs, as a paradigm-shifting technology, could be implemented as a large-area, tunable-density, and in situ reconfigurable electrophysiological mapping technology for personalized medicine in muscle treatments, myoelectric control, sports physiology, and human-machine interfaces.

### Materials and Methods

### Materials

Ag flakes (10 μm size, 99.9% trace metals basis, 327077), and poly(ethylene glycol)-block-poly(propylene glycol)-block-poly(ethylene glycol) (Pluronic P-123, 435465) were purchased from Sigma Aldrich and used without further modification. PEDOT:PSS (PH 1000) was from Ossila Limited. The insulation material (Pros-Aide) was a water-based acrylic emulsion from ADM Tronics. The conductive wire glue (made from graphite, polyvinyl acetate, and water) was from Anders Products.

### Conductive Ink Preparation.

The DoS conductive ink was prepared by first making the highly conductive PEDOT:PSS solution and then adding in the Ag flakes. First, the PEDOT:PSS solution was prepared by stirring 10 wt. % P-123 into the commercial PEDOT:PSS solution for 12 h at room temperature (˜22° C.) at 800 rpm. Afterward, the prepared solution was stored at ˜4° C. in a refrigerator. Prior to adding Ag flakes, the PEDOT:PSS solution was taken out of the refrigerator and stirred for 1-2 minutes. Then the corresponding amount of Ag flakes (1:2 weight ratio, Ag flakes: PEDOT:PSS solution) in the form of powder was added to the vial, and the PEDOT:PSS solution was added to the vial, and the mixture was stirred on a magnetic stirrer for about 1 h. The resulting ink was ready to use after the stirring, but it could be stirred more if any visible Ag flakes powder remained.

DoS MEA Fabrication on Skin with Custom Interconnection Schemes.

For interconnection schemes that were drawn (with or without a stencil) or when wires from a data acquisition (DAQ) system were directly attached to DoS electrodes, the approach described here was utilized. The DoS MEAs were prepared using modified ballpoint pens, stencils, the conductive ink, Pros-Aide, stainless steel wires (790900, A-M systems), conductive wire glue, electrode collar adhesive (TD23, Refa), and tape (Magic Tape, 3M). The fabrication of the stencils is described in. The skin of the subject was wiped with an alcohol prep pad for a few seconds, and the stencil was applied. If the stencil did not have interconnections, the electrodes were drawn into the circular parts of the stencil (see FIGS. 6A-6D). A stainless steel wire was laid on top of the electrode, and the electrode collar adhesive was laminated on top to connect the electrodes directly to the DAQ system. Finally, a drop of DoS ink was placed inside the hole of the electrode collar adhesive to sandwich the wire (FIGS. 7A-7B). If the stencil had interconnections, the first step was to draw the electrodes over the circular parts of the stencil (see FIGS. 6A-6D) and leave them to dry for 3-5 min. Next, the insulation material (Pros-Aide) was brushed onto the interconnect lines exposed in the stencil. After the insulation material became clear and slightly tacky (5-10 min), the interconnection lines were drawn over the Pros-Aide and left to dry another 3-5 min. The stencil was removed slightly before all the DoS ink appeared dry. A stainless steel wire was taped to the skin with the exposed part laying over the end of the DoS interconnect line to wire the electrodes to the DAQ. Conductive wire glue was painted onto two portions of the exposed wire over the interconnect (FIGS. 7A-7B) to clamp the wire down to the skin. A hairdryer (Conair) was held at a low setting for 1 min to cure the glue. Then one more layer was drawn with the DoS ink on top of the wire glue to sandwich the wire.

DoS MEA Fabrication on Skin with Prefabricated Interconnection Schemes.

The following approach was utilized if the interconnection scheme was prefabricated (e.g., in contexts when the design can be ascertained before the in situ application). The DoS MEAs were prepared using modified ballpoint pens, stencils, the conductive ink, Pros-Aide, and the prefabricated interconnection film. The skin of the subject was wiped with an alcohol prep pad for a few seconds, and the stencil was applied. The electrodes were drawn over the circular parts of the stencil (see FIGS. 6A-6D) and left to dry for 3-5 min. Next, the insulation material (Pros-Aide) was brushed onto the interconnect lines exposed in the stencil. After the insulation material became clear and slightly tacky (5-10 min), the interconnection lines were drawn over the Pros-Aide and left to dry another 3-5 min. The stencil was removed slightly before all the DoS ink appeared dry. Prior to applying the prefabricated interconnection film to the skin, the exposed PI film and unused interconnection lines were covered with Pros-Aide. After 5-10 min (the film appeared clear and was slightly tacky), the interconnection film was laminated to the skin with the interconnection film aligned to the DoS interconnection lines. The fabrication of the interconnection film is described in the following. A glass slide was cleaned using acetone, isopropyl alcohol (IPA), and DI water. A ˜2 μm thick polyimide (PI-2545, HD Microsystems) film was spin coated on the glass slide. Then 5 nm/100 nm thick Cr/Au layers were deposited via an e-beam evaporator. The metal layers were then patterned by photolithography and wet etching. The film was released from the glass slide using buffered oxide etchant (BOE, 6:1, Transene Company Inc.). After releasing, the metal interconnect was connected to a custom-made PCB through an ACF cable for data measurement. The PCB had male headers that could be attached to a DAQ system with breadboard wires.

### Skin-Electrode Impedance Characterization.

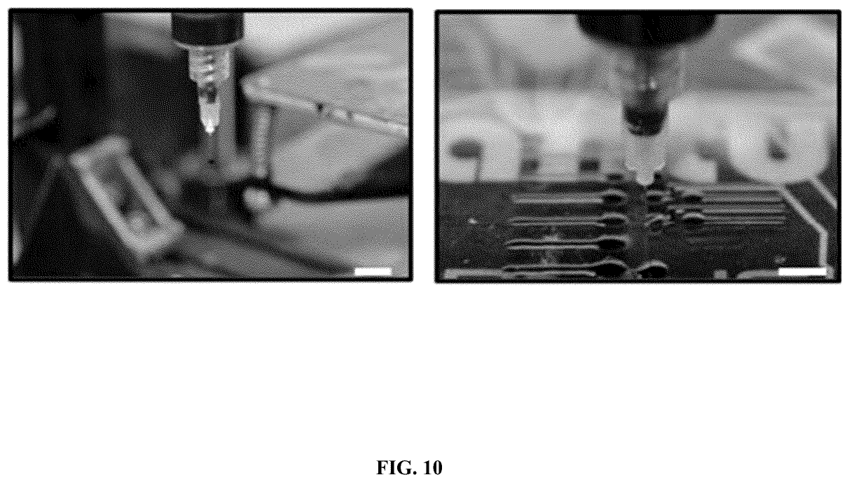

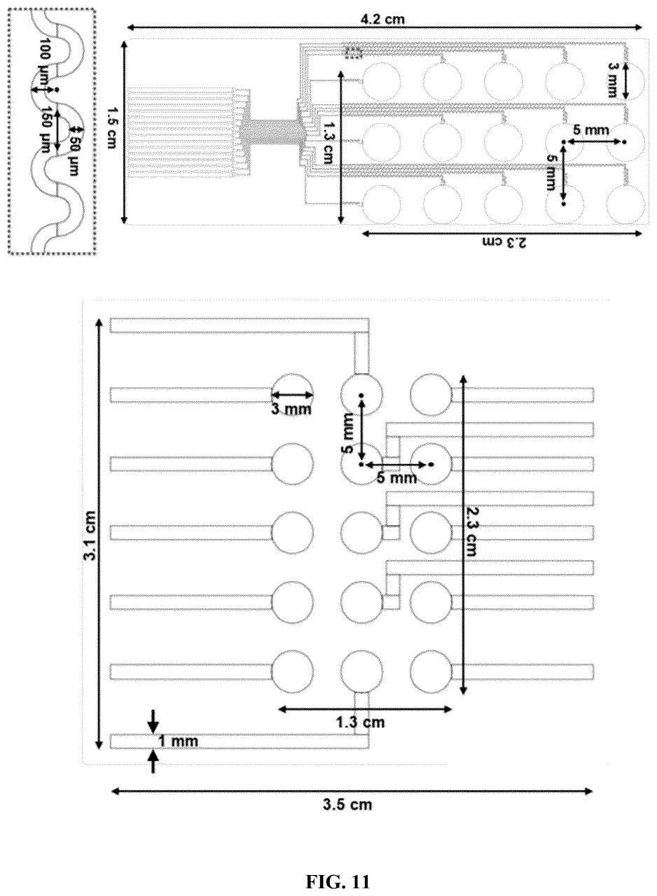

All the procedures were approved by the Institutional Review Board of the University of Houston, TX (USA) and informed consent was obtained (Protocol 2765). To validate the sensing capabilities of the DoS MEAs, they were compared with multiple types of the existing bioelectronics and the conventional technology. Specifically, we first compared the impedance characteristics of the DoS electrodes with those of a structurally engineered stretchable Au mesh-based MEA, a 3D printed and intrinsically stretchable PEDOT:PSS MEA, and a flexible printed TMSi grid. The fabrication processes of the stretchable Au mesh and printed PEDOT:PSS-based MEAs are depicted in FIG. 9 and FIG. 10, respectively. The dimensions of the arrays are labeled in FIG. 11. It should be noted that the number of electrodes, diameters, and interelectrode distance was consistent among the wearable MEAs, apart from those of the FPC grid. Specifically, the DoS, PEDOT:PSS, and Au MEAs each had electrodes that were 3 mm in diameter and spaced 5 mm apart, all arranged in a 3×5 (row×column) grid. The interelectrode spacing is reported center-to-center throughout the rest of this work. A custom connection scheme was developed to ensure that all devices were evaluated in a similar manner (FIG. 13) to acquire data specifically from the FPC grid. The only additional connection needed for the SEI measurements from the FPC grid was a proprietary TMSi cable that connected to the contact pads of the conventional grid and an adapter that was fitted with breadboard wires. It should be noted that the FPC grid had electrodes that were 4.5 mm in diameter and had an interelectrode distance of 8.75 mm. An SEI heatmap of a 3×5 portion of the 64-channel FPC grid is shown in FIG. 14. For all SEI measurements, the same measurement settings were used. The SEI was measured using an impedance analyzer (Multi/Autolab M204, Metrohm) that measured the impedance from 1000 to 1 Hz. A two-electrode configuration was used. The working electrode and reference electrode were connected to two electrodes in the array placed on the skin. In each row (for each type of MEA), one electrode was fixed as the reference electrode, and the working electrode was moved to another electrode (of the MEA) sequentially to create the SEI heatmaps.

### EMG Data Acquisition.

The areas of skin on which the electrodes were placed were prepared with an alcohol prep pad that was scrubbed on the skin for a few seconds. It is noted that this preparation step is not always necessary to successfully capture EMG signals. The snap electrical leads were connected to an interface board (Recording Controller, Intan Technologies) via an amplifier board (RHD2132, Intan Technologies) with unipolar input channels. In the DAQ program, a sampling rate of 2000 Hz was utilized, and the notch filter (60 Hz) setting was turned on. A wet cuff electrode was placed on the bony portion of the wrist to serve as the ground electrode for all measurements. The bandwidth was set to 0.1-1000 Hz. Signals were processed with a third-order Butterworth bandpass filter, with the cutoff frequencies being 20 and 500 Hz.

### Skin Deformation-Induced Motion During EMG Sensing.

The quality of the EMG signals from each MEA was determined without and with skin deformation-induced motion for the three subjects. The subjects were asked to squeeze their right hand into a fist at regular intervals, three times per trial (n=10, per MEA type). After an initial flexion, their skin was manually deformed by the experimenter (at a speed of ˜2 mm/s) at opposite ends of the MEAs during the following two flexions as an extreme case of skin deformation during muscle contraction. In the second flexion, the skin around the MEA was stretched (stretching motion duration is indicated by the dark bar) and released (indicated by the light bar). In the third contraction, the skin around the MEA was compressed and released. Signals were processed with a third-order Butterworth bandpass filter, with the cutoff frequencies being 1 and 500 Hz. The lower cutoff is used here to demonstrate the effect of the induced motion.

### Innervation Zone Localization and Motor Unit Action Potential Detection.

Electrodes were drawn into a 4×8 grid, each being 4.5 mm in diameter and spaced 8.75 mm apart to match the dimensions of the commercial FPC grid (FIG. 17) for further comparisons. For each row of the grid, a propagation map was created, and innervation zones could easily be identified through the changing direction of the deflection from the baseline. Bipolar EMG signals were first derived from the monopolar signals by taking the difference between neighboring channels along the muscle fiber direction. The most prominent motor unit action potentials present in the surface interferential pattern were separated via blind source separation using the Joint Approximation Diagonalization of Eigenmatrices (JADE) algorithm. Separated components demonstrating motor unit spiking activity were selected, and spike times were determined via threshold detection to isolate spikes generated from a single motor unit. Time series EMG data were then spike-triggered averaged with respect to the determined spike-times to give the spatiotemporal representation of the motor unit action potential, as shown in FIG. 3B. To achieve decomposition of lower amplitude motor unit action potentials, the K-Means Clustering and Convolution Kernel Compensation (KMCKC) algorithm was utilized to determine spike times from a more diverse pool of motor units. Similarly, the time-series EMG data were spike-trigger averaged for each decomposed MUAP, and represented spatially, as shown in FIG. 3B. The IZs were determined by inspecting the channel where propagating MUAPs demonstrate a phase reversal.

### EMG Measurement During Seated Resistance Band Curls.

Each of the three subjects was asked to perform seated resistance band bicep curls while wearing a 4×4 DoS MEA and FPC grid (FIG. 3D) for ˜30 min with rests in between. A metronome was set to 50 bpm for 1 min sets, with 1 min breaks between sets until each fifth set. About 25 contraction/relaxation cycles were performed in each set. In the fifth and tenth sets, the subjects were given 2 min breaks. Fewer rows and channels (again in a 4×4 format) were used from the 64-channel FPC grid with a 17.5 mm spacing between electrodes for both MEAs to simplify the data processing. Anatomically, the long and short heads of the biceps brachii were covered by both MEAs, and the centers of the muscles slid back and forth under different regions of the MEAs. Only the contraction regions of the EMG data were used for processing. The plotted data shows the averaged SNR per row for each contraction over time. Signals were processed with a third-order Butterworth bandpass filter, with cutoff frequencies at 20 and 500 Hz.

### Reconfigurable DoS MEA and Conventional Grid Setup.

For all the EMG measurements performed during hand gesture experiments throughout this work, the subjects rested their arm on a table with their hand hanging slightly off but kept their hands and wrists in a neutral position to minimize any pronation/supination based artifacts. The FPC grid was placed on the belly of the flexor muscles in the forearm of three subjects, and representative results are shown. The DoS MEAs were drawn in arrangement 1 (FIG. 4A), and the data was obtained simultaneously with the FPC grid and DoS MEAs. It is assumed that the individual applying the DoS electrodes to the patient has a general understanding of the muscle anatomy and can simply fine-tune the DoS MEA by adding and removing electrodes. After analyzing the data and noting that the extent of the muscle activity seemed to extend beyond the analyzed anatomical location, another arrangement was tested. The unnecessary electrodes of the DoS MEA were wiped off with a wet cotton swab/paper towel, and DoS electrodes were drawn onto the new locations for arrangement 2. Again, after analyzing the data for arrangement 2, it seemed that activity extended in the distal direction. Arrangement 3 was made to accommodate this, in the same manner performed for arrangement 2.

### Customized DoS MEA Fabrication for Finger Gesture Classification.

Stencils were designed for custom DoS MEAs that covered the entire circumference of the forearm at four positions (FIG. 5A and FIG. 23). Each stencil was a linear array (containing eight electrodes) with different lengths and varied spacing based on the circumference at the four positions, which were evenly distributed over the bellies of the forearm muscle groups.

Finger gesture classification and principal component analysis. For each subject, four sessions per gesture were performed, with each session having 10 trials. Representative excitation feature maps are shown in FIG. 5C, specifically using the root-mean-square voltage (Vᵣₘₛ) as the feature. The Vᵣₘₛ feature was extracted from the steady-state portion of four unique finger flexion gestures performed by the participants from both the DoS MEAs and commercial FPC grids. Vᵣₘₛ features from all finger gestures were standardized to zero mean and unit variance (Z-score) prior to principal component analysis (PCA) for visualization of cluster separation in reduced dimensions. Vᵣₘₛ features were then classified via a Linear Discriminant Analysis (LDA) model. The LDA model performance was evaluated using 5-fold validation to derive the cross-validated classification loss.

### Prosthetic Hand Control.

After offline analysis and classification were performed, the same LDA classifier was used to perform online predictions based on a model trained with the obtained data from the subjects. The output of the classification was sent to a custom Arduino script, which was written to control the prosthetic hand. EMG data was obtained in near real-time from the DoS MEAs customized to each subject who performed the gestures.

### Ballpoint Pen Preparation.

Ballpoint pens (557154012, PEN+GEAR) were fully disassembled. The balls from the pen tips and the original inks were removed. The tips and ink barrels were thoroughly cleaned in acetone, sonicated in deionized (DI) water, and air dried. Then, the ink was injected into the emptied ink barrels via a syringe and 26-gauge needle.

### Stencil Fabrication.

The stencils were designed in AutoCAD. A cutting board was layered with one layer of packing tape (Duck). The cutting machine (Silhouette Cameo) was programmed to cut the stencils based on the designs. The stencils were removed from the cutting board and then placed onto a sticker sheet for later use.

### Fabrication of Stretchable Au MEA.

First, a glass slide was cleaned using acetone, isopropyl alcohol (IPA), and DI water. A 200-250 nm thick polyimide (PI-2545, HD Microsystems) film was made by spin coating. Then 5 nm/100 nm thick Cr/Au layers were deposited via an e-beam evaporator. The metal layers were then patterned by photolithography and wet etching. The PI was patterned by reactive ion etching (RIE, Oxford Plasma Lab 80 Plus). Finally, a layer of poly(methyl methacrylate) (PMMA) was spin coated onto the metal side to aid transfer and temporarily maintain the structure of the electrode. The electrode was released from the glass using buffered oxide etchant (BOE, 6:1, Transene Company Inc.) and then picked up using wax paper. The PMMA was dissolved using acetone. The electrode was then transferred from the wax paper to the skin.

### DoS MEA Interconnection Setup for Data Acquisition.

Unlike the typical bioelectronics, the DoS sensors and devices present the unique opportunity to make interconnection systems directly on the body using just the DoS inks. For the purposes of this work, we demonstrate wired approaches to illustrate the potential use of DoS electrode arrays in a simple manner. Using an electrode collar adhesive can allow the user to ascertain that the stainless-steel wires directly contact the DoS electrodes (FIG. 7A). The electrode collar is donut-shaped and the hole in the center allows the experimenter to confirm the wires are secured to the DoS electrodes. The cross-sectional view of the interface is shown at the bottom of FIG. 7A. This approach could be adapted to DoS interconnects, if the spacing between them is relatively large (>10 mm). Furthermore, the use of conductive adhesives can also facilitate contact between external wires and the DoS interconnects in MEAs as shown in FIG. 7B. Here we chose to use conductive wire glue as it is water-based, safe for use on the skin, and can be dried quickly. In this alternative approach, after the interconnect was drawn, the conductive wire was secured at two ends with a conductive glue (Wire Glue, Anders Products). The wire was then covered with additional DoS conductive ink across the entire length exposed to the interconnect. The cross-section of this arrangement is shown in the bottom FIG. 7B as well. For use cases in which the array is customized to the individual or the interconnection design is uncertain, the adhesive and external wire approaches are suitable. If the design of the array interconnection is known beforehand however, interconnection films could be custom manufactured through traditional microfabrication as well. An example of this is shown in FIG. 8. The interconnection film was designed in such a pattern that it could be adapted to either side of the DoS MEA. It is noted that although extra interconnection lines were fabricated on the film, their corresponding contact pads did not have any connection to the data acquisition (DAQ) system. This approach could be used to rapidly collect data from several electrodes (tens of channels) simultaneously as the interconnecting film could be prepared with an anisotropic conductive film (ACF) cable bonded to a printed circuit board (PCB). The PCB could be connected to any data DAQ with an adapter. Depending on whether the interconnect design is predetermined, any of the aforementioned approaches would suffice for collecting data from several channels of DoS electrodes.

Fast Fourier Transform Characteristics of EMG Data from MEAs.

The fast Fourier Transforms (FFT) of the averaged EMG signals (initial contraction) from each array are shown in FIG. 16. The DoS and PEDOT:PSS MEAs show a similar magnitude through the lower half of the analyzed spectrum (1-500 Hz), while the PEDOT:PSS MEA shows a slightly higher magnitude in frequencies above 150 Hz. This is likely due to the higher concentration of PEDOT:PSS as the electrode material and better ionic conductivity compared to the high concentration of Ag flakes used in the DoS ink. The Au MEA also shows a similar frequency profile to that of the DoS MEA, except that the magnitude remains relatively lower below 150 Hz. The frequency profile of the FPC grid shows relatively higher power across the entire spectrum compared to the other MEAs, which is likely due to the application of a conductive gel to the surface of the grid prior to placement on the target muscle. For prosthetic control, wearable grids may be worn for hours, to days, weeks, or months, making gels a generally undesirable additive since they can dry out quickly.

### Muscle Fiber Propagation Speed and Detection.

To calculate the muscle fiber conduction velocity, first, the differences in timing of the sequential positive (or negative) peaks across the columns in each row (A, B, C, D) from the propagation maps (FIGS. 18A-18E) were determined. The distance between each electrode was divided by each of the differences and the values were averaged and converted to m/s. The number of motor units captured using the DoS MEA and FPC grid (placed on the same muscle, were compared. The number of motor units for a representative subject detected using the DoS MEA and FPC grid were 3 and 17, respectively. This difference in the number of motor units detected could be due to the highly optimized and locally shielded data acquisition interface used with the FPC grid as compared to the current unshielded data acquisition approach with DoS electronics. Another important consideration for MUAP detection is that by knowingly targeting the belly of the muscle, the number of electrodes required could be reduced, which reduces the computation performed in postprocessing.

### SNR Calculation for EMG Signals.

To calculate the SNR for each of the sensor types, first the power spectral density estimate was obtained using Welch's method in MATLAB. The parameters for the pwelch function were chosen to be a 400-point Hanning window and a 50% overlap. Signals in the 20-500 Hz range of the power spectrum represent the “signal” in the SNR calculation and the power was summed over those frequencies and normalized to be in units of dB. The noise was averaged from the rest of the power spectrum (500-1000 Hz) and represent the “noise” in the SNR calculation. The following formula was used to convert the ratio of the signal and noise to power in dB:

$$SNR = 10 * \frac{{P}_{( s )}}{{P}_{( n )}}$$

where P₍ₛ₎ is the power of the signal and P₍ₙ₎ is the power of the noise.

### REFERENCES

The following references are incorporated herein by reference in their entireties.
Milosevic B, Benatti S, Farella E (2017) Design challenges for wearable EMG applications. Design, Automation & Test in Europe Conference & Exhibition (DATE), 2017, pp 1432-1437.
Drost G, Stegeman D F, van Engelen B G, Zwarts M J (2006) Clinical applications of high-density surface EMG: a systematic review. J Electromyogr Kinesiol 16:586-602.
Afsharipour B, Soedirdjo S, Merletti R (2019) Two-dimensional surface EMG: The effects of electrode size, interelectrode distance and image truncation. Biomedical Signal Processing and Control 49:298-307.
Vigotsky A D, Halperin I, Lehman G J, Trajano G S, Vieira T_{M} (2017) Interpreting Signal Amplitudes in Surface Electromyography Studies in Sport and Rehabilitation Sciences. Front Physiol 8:985.
Jahanmiri-Nezhad F, Barkhaus P E, Rymer W Z, Zhou P (2015) Innervation zones of fasciculating motor units: observations by a linear electrode array. Front Hum Neurosci 9:239.
Topalovic I, Graovac S, Popovic D B (2019) EMG map image processing for recognition of fingers movement. J Electromyogr Kinesiol 49:102364.
Lara J E, Cheng L K, Rohrle O, Paskaranandavadivel N (2022) Muscle-Specific High-Density Electromyography Arrays for Hand Gesture Classification. IEEE Trans Biomed Eng 69:1758-1766.
Zhu M, Zhang H, Wang X, Wang X, Yang Z, Wang C, Samuel O W, Chen S, Li G (2021) Towards optimizing electrode configurations for silent speech recognition based on high-density surface electromyography. J Neural Eng 18.
Michelsen J S, Lund M C, Alkjaer T, Finni T, Nielsen J B, Lorentzen J (2020) Wearable electromyography recordings during daily life activities in children with cerebral palsy. Dev Med Child Neurol 62:714-722.
Daley H, Englehart K, Hargrove L, Kuruganti U (2012) High density electromyography data of normally limbed and transradial amputee subjects for multifunction prosthetic control. J Electromyogr Kinesiol 22:478-484.
Farrell T R, Weir R F (2008) A comparison of the effects of electrode implantation and targeting on pattern classification accuracy for prosthesis control. IEEE Trans Biomed Eng 55:2198-2211.
Favieiro G, Cene V H, Balbinot A (2016) Self-adaptive Method for sEMG Movement Classification Based on Continuous Optimal Electrode Assortment. Brazilian Journal of Instrumentation and Control 4.
Jordanic M, Rojas-Martinez M, Mananas M A, Alonso J F (2016) Spatial distribution of HD-EMG improves identification of task and force in patients with incomplete spinal cord injury. J Neuroeng Rehabil 13:41.
Stango A, Negro F, Farina D (2015) Spatial correlation of high density EMG signals provides features robust to electrode number and shift in pattern recognition for myocontrol. IEEE Trans Neural Syst Rehabil Eng 23:189-198.
Tam S, Boukadoum M, Campeau-Lecours A, Gosselin B (2021) Intuitive real-time control strategy for high-density myoelectric hand prosthesis using deep and transfer learning. Sci Rep 11:11275.
Roche A D, Lakey B, Mendez I, Vujaklija I, Farina D, Aszmann O C (2019) Clinical Perspectives in Upper Limb Prostheses: An Update. Current Surgery Reports 7.
Roche A D, Rehbaum H, Farina D, Aszmann O C (2014) Prosthetic Myoelectric Control Strategies: A Clinical Perspective. Current Surgery Reports 2.
Liu J, Zhou P (2013) A novel myoelectric pattern recognition strategy for hand function restoration after incomplete cervical spinal cord injury. IEEE Trans Neural Syst Rehabil Eng 21:96-103.
Liu J, Li X, Li G, Zhou P (2014) EMG feature assessment for myoelectric pattern recognition and channel selection: a study with incomplete spinal cord injury. Med Eng Phys 36:975-980.
Yao B, Peng Y, Zhang X, Zhang Y, Zhou P, Pu J (2020) The influence of common component on myoelectric pattern recognition. J Int Med Res 48:300060520903617.
Pan L, Zhang D, Jiang N, Sheng X, Zhu X (2015) Improving robustness against electrode shift of high density EMG for myoelectric control through common spatial patterns. J Neuroeng Rehabil 12:110.
Young A J, Hargrove L J, Kuiken T A (2011) The effects of electrode size and orientation on the sensitivity of myoelectric pattern recognition systems to electrode shift. IEEE Trans Biomed Eng 58:2537-2544.
Schlink B R, Nordin A D, Ferris D P (2020) Comparison of Signal Processing Methods for Reducing Motion Artifacts in High-Density Electromyography During Human Locomotion. IEEE Open Journal of Engineering in Medicine and Biology 1:156-165.
Roland T (2020) Motion Artifact Suppression for Insulated EMG to Control Myoelectric Prostheses. Sensors (Basel) 20.
Afanasenkau D, Kalinina D, Lyakhovetskii V, Tondera C, Gorsky O, Moosavi S, Pavlova N, Merkulyeva N, Kalueff A V, Minev I R, Musienko P (2020) Rapid prototyping of soft bioelectronic implants for use as neuromuscular interfaces. Nat Biomed Eng 4:1010-1022.
Chandra S, Li J, Afsharipour B, Cardona A F, Suresh N L, Tian L, Deng Y, Zhong Y, Xie Z, Shen H, Huang Y, Rogers J A, Rymer W Z (2020) Performance Evaluation of a Wearable Tattoo Electrode Suitable for High-Resolution Surface Electromyogram Recording. IEEE Trans Biomed Eng PP.
Gandla S, Chae H J, Kwon H-j, Won Y C, Park H, Lee S, Song J, Baek S, Hong Y-D, Kim D H, Kim S (2021) Ultrafast prototyping of large-area stretchable electronic systems by laser ablation technique for controllable robotic arm operations. IEEE Transactions on Industrial Electronics:1-1.
Jin H, Matsuhisa N, Lee S, Abbas M, Yokota T, Someya T (2017) Enhancing the Performance of Stretchable Conductors for E-Textiles by Controlled Ink Permeation. Advanced Materials 29.
Kwon Y T, Kim Y S, Kwon S, Mahmood M, Lim H R, Park S W, Kang S O, Choi J J, Herbert R, Jang Y C, Choa Y H, Yeo W H (2020) All-printed nanomembrane wireless bioelectronics using a biocompatible solderable graphene for multimodal human-machine interfaces. Nat Commun 11:3450.
Kwon Y T, Norton J J S, Cutrone A, Lim H R, Kwon S, Choi J J, Kim H S, Jang Y C, Wolpaw J R, Yeo W H (2020) Breathable, large-area epidermal electronic systems for recording electromyographic activity during operant conditioning of H-reflex. Biosens Bioelectron 165:112404.
Moin A, Zhou A, Rahimi A, Menon A, Benatti S, Alexandrov G, Tamakloe S, Ting J, Yamamoto N, Khan Y, Burghardt F, Benini L, Arias A C, Rabaey J M (2020) A wearable biosensing system with in-sensor adaptive machine learning for hand gesture recognition. Nature Electronics 4:54-63.
Tian L, Zimmerman B, Akhtar A, Yu K J, Moore M, Wu J, Larsen R J, Lee J W, Li J, Liu Y, Metzger B, Qu S, Guo X, Mathewson K E, Fan J A, Cornman J, Fatina M, Xie Z, Ma Y, Zhang J, Zhang Y, Dolcos F, Fabiani M, Gratton G, Bretl T, Hargrove L J, Braun P V, Huang Y, Rogers J A (2019) Large-area MRI-compatible epidermal electronic interfaces for prosthetic control and cognitive monitoring. Nature Biomedical Engineering 3:194-205.
Wang Y, Yin L, Bai Y, Liu S, Wang L, Zhou Y, Hou C, Yang Z, Wu H, Ma J, Shen Y, Deng P, Zhang S, Duan T, Li Z, Ren J, Xiao L, Yin Z, Lu N, Huang Y (2020) Electrically compensated, tattoo-like electrodes for epidermal electrophysiology at scale. Sci Adv 6.
Xu Y, Sun B, Ling Y, Fei Q, Chen Z, Li X, Guo P, Jeon N, Goswami S, Liao Y, Ding S, Yu Q, Lin J, Huang G, Yan Z (2020) Multiscale porous elastomer substrates for multifunctional on-skin electronics with passive-cooling capabilities. Proc Natl Acad Sci USA 117:205-213.
Xu Y, Zhao G, Zhu L, Fei Q, Zhang Z, Chen Z, An F, Chen Y, Ling Y, Guo P, Ding S, Huang G, Chen P Y, Cao Q, Yan Z (2020) Pencil-paper on-skin electronics. Proc Natl Acad Sci USA 117:18292-18301.
Ershad F, Thukral A, Yue J, Comeaux P, Lu Y, Shim H, Sim K, Kim N I, Rao Z, Guevara R, Contreras L, Pan F, Zhang Y, Guan Y S, Yang P, Wang X, Wang P, Wu X, Yu C (2020) Ultra-conformal drawn-on-skin electronics for multifunctional motion artifact-free sensing and point-of-care treatment. Nat Commun 11:3823.
Patel S, Ershad F, Lee J, Chacon-Alberty L, Wang Y, Morales-Garza M A, Haces-Garcia A, Jang S, Gonzalez L, Contreras L, Agarwal A, Rao Z, Liu G, Efimov I R, Zhang Y S,
Zhao M, Isseroff R R, Karim A, Elgalad A, Zhu W, Wu X, Yu C (2022) Drawn-on-Skin Sensors from Fully Biocompatible Inks toward High-Quality Electrophysiology. Small.
Kim D, Nicoletti C, Soedirdjo S D H, Baghi R, Garcia M G, Laubli T, Wild P, BotterA, Martin B J (2021) Effect of Periodic Voluntary Interventions on Trapezius Activation and Fatigue During Light Upper Limb Activity. Hum Factors:187208211050723.
Gallina A, Merletti R, Gazzoni M (2013) Uneven spatial distribution of surface EMG: what does it mean?Eur J Appl Physiol 113:887-894.
Campanini I, Merlo A, Disselhorst-Klug C, Mesin L, Muceli S, Merletti R (2022) Fundamental Concepts of Bipolar and High-Density Surface EMG Understanding and Teaching for Clinical, Occupational, and Sport Applications: Origin, Detection, and Main Errors. Sensors (Basel) 22.
Inzelberg L, David-Pur M, Gur E, Hanein Y (2020) Multi-channel electromyography-based mapping of spontaneous smiles. J Neural Eng 17:026025.
De Luca C J, Gilmore L D, Kuznetsov M, Roy S H (2010) Filtering the surface EMG signal: Movement artifact and baseline noise contamination. J Biomech 43:1573-1579.
Okajima S, Ianez E, Yamasaki H, Garcia A C, Alnajjar F S, Hattori N, Shimoda S (2019) Quantification of Extent of Muscle-skin Shifting by Traversal sEMG Analysis Using High-density sEMG Sensor. 272-277.
Huang C, Klein C S, Meng Z, Zhang Y, Li S, Zhou P (2019) Innervation zone distribution of the biceps brachii muscle examined using voluntary and electrically-evoked high-density surface EMG. J Neuroeng Rehabil 16:73.
Felici F, Del Vecchio A (2020) Surface Electromyography: What Limits Its Use in Exercise and Sport Physiology?Front Neurol 11:578504.
Liu Y, Zhang C, Dias N, Chen Y T, Li S, Zhou P, Zhang Y (2020) Transcutaneous innervation zone imaging from high-density surface electromyography recordings. J Neural Eng 17:016070.
Celadon N, Dosen S, Binder I, Ariano P, Farina D (2016) Proportional estimation of finger movements from high-density surface electromyography. Journal of NeuroEngineering and Rehabilitation 13:73.
Olsson A E, Sager P, Andersson E, Bjorkman A, Malesevic N, Antfolk C (2019) Extraction of Multi-Labelled Movement Information from the Raw HD-sEMG Image with Time-Domain Depth. Sci Rep 9:7244.
Leijnse I N, Campbell-Kyureghyan N H, Spektor D, Quesada P M (2008) Assessment of individual finger muscle activity in the extensor digitorum communis by surface EMG. J Neurophysiol 100:3225-3235.
Tsujimura T, Yamamoto S, Izumi K (2012) Hand Sign Classification Employing Myoelectric Signals of Forearm. Computational Intelligence in Electromyography Analysis—A Perspective on Current Applications and Future Challenges).
Hu X, Suresh N L, Xue C, Rymer W Z (2015) Extracting extensor digitorum communis activation patterns using high-density surface electromyography. Front Physiol 6:279.
Dai C, Hu X (2019) Extracting and Classifying Spatial Muscle Activation Patterns in Forearm Flexor Muscles Using High-Density Electromyogram Recordings. Int J Neural Syst 29:1850025.
Parajuli N, Sreenivasan N, Bifulco P, Cesarelli M, Savino S, Niola V, Esposito D, Hamilton T J, Naik G R, Gunawardana U, Gargiulo G D (2019) Real-Time EMG Based Pattern Recognition Control for Hand Prostheses: A Review on Existing Methods, Challenges and Future Implementation. Sensors (Basel) 19.
Ellis R J, Zhu B, Koenig J, Thayer J F, Wang Y (2015) A careful look at ECG sampling frequency and R-peak interpolation on short-term measures of heart rate variability. Physiol Meas 36:1827-1852.
Wang J, Tang L, Bronlund J E (2013) Surface EMG signal amplification and filtering. International Journal of Computer Applications 82.
Cardoso J F, Souloumiac A (1993) Blind beamforming for non-gaussian signals. IEE Proceedings F Radar and Signal Processing 140.
Ning Y, Zhu X, Zhu S, Zhang Y (2015) Surface EMG decomposition based on K-means clustering and convolution kernel compensation. IEEE J Biomed Health Inform 19:471-477.
Gazzoni M, Celadon N, Mastrapasqua D, Paleari M, Margaria V, Ariano P (2014) Quantifying forearm muscle activity during wrist and finger movements by means of multi-channel electromyography. PLoS One 9:e109943.
Bihar E, Roberts T, Zhang Y, Ismailova E, Herve T, Malliaras G G, De Graaf J B, Inal S, Saadaoui M (2018) Fully printed all-polymer tattoo/textile electronics for electromyography. Flexible and Printed Electronics 3:034004.
Kwon Y T, Norton J J S, Cutrone A, Lim H R, Kwon S, Choi J J, Kim H S, Jang Y C, Wolpaw J R, Yeo W H (2020) Breathable, large-area epidermal electronic systems for recording electromyographic activity during operant conditioning of H-reflex. Biosens Bioelectron 165:112404.
Dai C, Hu X (2019) Extracting and Classifying Spatial Muscle Activation Patterns in Forearm Flexor Muscles Using High-Density Electromyogram Recordings. Int J Neural Syst 29:1850025.

What is claimed is:

1. A kit for preparation of a network of drawn-on sensors, the kit comprising:
electrically conductive ink configured to adhere to a surface and form an electrode and an interconnect when applied to the surface;
an ink applicator configured to apply the electrically conductive ink to the surface;
an insulative material applicator configured to apply an electrically insulative material to the surface; and
an electrical contact configured to place the interconnect in electrical connection with a data acquisitioning system.

2. The kit of claim 1, wherein:
the ink applicator is a pen, a brush, a dispensing device, and/or a printer device; and
the insulative material applicator is a pen, a brush, a dispensing device, and/or a printer device.

3. The kit of claim 1, wherein:
the surface is skin of an animal, skin of a human, or a surface of artificial or synthetic skin.

4. The kit of claim 1, further comprising:
a stencil configured to be placed against the surface and guide application of the electrically conductive ink and/or the insulative material.

5. The kit of claim 1, further comprising:
conductive glue configured to adhere the electrical contact to the surface.

6. The kit of claim 1, wherein:
the electrical contact includes an electrically conductive wire, an electrically conductive film, and/or an electrically conductive pad.

7. The kit of claim 1, wherein:
the electrical contact comprises an anisotropic material.

8. The kit of claim 1, further comprising:
the data acquisitioning system.

9. The kit of claim 1, wherein:
the electrically conductive ink includes an Ag/poly(3,4-ethylenedioxythiophene)-poly(styrenesulfonate) (“Ag-PEDOT:PSS”) composite; and
the electrically insulative material includes a water-based acrylic emulsion.

10. A method for fabricating an electrode ink, the method comprising:
preparing a poly(3,4-ethylenedioxythiophene)-poly(styrenesulfonate) (“PEDOT:PSS”) solution;
adding Ag flakes to the solution in a 1:2 weight ratio of Ag flakes to PEDOT:PSS solution to form a Ag-PEDOT:PSS composite; and
stirring and/or agitating the Ag-PEDOT:PSS composite.

11. A method for generating a sensor network, the method comprising:
applying an electrically conductive ink to a surface of skin to form an electrode point;
applying the electrically insulative material to the surface of skin;
applying the electrically conductive ink to the insulative material to form an interconnect extending from an electrode point;
placing an electrical contact in electrical connection with the interconnect, wherein the electrical contact is configured to be placed in electrical connection with a data acquisitioning system.

12. The method of claim 11, further comprising:
placing the electrical contact in electrical connection with the data acquisitioning system.

13. The method of claim 12, further comprising:
monitoring, measuring, and/or sensing electrical activity of the electrode;
wherein the electrical activity includes voltage, a change in voltage, current, a change in current, impedance, and/or a change in impedance.

14. The method of claim 13, further comprising:
performing electromyography; and/or
determining a movement, a gesture, a muscle activation, and/or a muscle relaxation based on the electrical activity.

15. The method of claim 14, further comprising:
measuring muscle response or electrical activity in response to a nerve's stimulation of a muscle.

16. The method of claim 15, further comprising:
detecting a neuromuscular abnormality.

17. The method of claim 14, further comprising:
translating the movement, the gesture, the muscle activation, and/or the muscle relaxation to an actuation signal configured to be transmitted to a prosthetic, a robotic prosthetic, or a gesture-controlled robot.

18. The method of claim 11, further comprising:
allowing or forcing flexure of the skin.

19. The method of claim 18, wherein:
the flexure of the skin induces strain on the electrically conductive ink to cause elastic deformation of the electrically conductive ink.

20. The method of claim 11, further comprising:
forming a multielectrode array or a network of sensors on the surface of skin by applying plural electrodes and plural interconnects in an arrangement.

21. The method of claim 11, wherein:
the skin is animal skin, human skin, or artificial or synthetic skin.

22. A method for creating at least one sensor on skin, the method comprising:
applying an electrically conductive ink to a surface of skin to form an electrical circuit;
wherein the skin is animal skin, human skin, or artificial or synthetic skin.

23. The method of claim 22, wherein:
signals generated from the electrical circuit are artifact-free.
